(19) United States
(12) Patent Application Publication
(10) Pub. No.: US 2023/0410940 A1
(43) Pub. Date: Dec. 21, 2023

(54) ENGINEERED BACTERIAL CELLS AND METHODS OF PRODUCING THE SAME

(71) Applicant: UCHICAGO ARGONNE, LLC, Chicago, IL (US)

(72) Inventors: Michael Fonstein, Burr Ridge, IL (US); Paul D. Hanke, Urbana, IL (US); Bruce Parrello, Chicago, IL (US); Rick Stevens, Chicago, IL (US); Thomas S. Brettin, Farragut, TN (US); Christopher S. Henry, Wilmette, IL (US)

(73) Assignee: UCHICAGO ARGONNE, LLC, Chicago, IL (US)

(21) Appl. No.: 18/115,412

(22) Filed: Feb. 28, 2023

Related U.S. Application Data
(60) Provisional application No. 63/315,340, filed on Mar. 1, 2022.

(51) Int. Cl. G16B 20/00; C12N 1/20

(52) U.S. Cl. CPC G16B 20/00; C12R 2001/19; C12N 1/20

(57) ABSTRACT

The present disclosure provides novel engineered target-biomolecule-producing bacterial strains and methods of producing the same. To engineer bacterial strains capable of producing substantial levels of a target biomolecule, the methods may implement the use of metabolic modeling and machine learning methods. The methods and bacterial strains produced by the methods may be implemented in further optimizing a biosynthetic pathway, e.g., to improve the production of a target biomolecule of interest, e.g., an amino acid, such as threonine.

### CROSS-REFERENCE TO RELATED APPLICATION

This application claims the benefit of U.S. Provisional Patent Application Ser. No. 63/315,340, filed Mar. 1, 2022, the entire contents of which is incorporated herein by reference.

### GOVERNMENT RIGHTS

This invention was made with government support under Contract No. DE-AC02-06CH11357 awarded by the United States Department of Energy to UChicago Argonne, LLC, operator of Argonne National Laboratory. The government has certain rights in the invention.

### TECHNICAL FIELD

The present invention relates to the fields of microbiology, microbial genetics, synthetic biology and computational biology. More specifically, the invention relates to novel bacterial strains, methods of producing novel bacterial strains, and processes employing these strains for the large scale production of biomolecules, including amino acids such as threonine.

### BACKGROUND

While microorganism-based biomanufacturing continues to be developed and improved, including as relates to threonine production, the development of new strains or improvement to production from existing strains is limited by a number of factors. First, current methods depend on extensive and specific knowledge, which may not exist for many desirable products. Second, there are a large number of factors affecting product yield, which make exhaustive experimental testing infeasible. Third, use of a reductionist approach (e.g., testing one strain modification at a time), attractive for its simplicity in a complex environment often hide effects that can be seen only in combinations of such modifications.

While these are known limitations in biomanufacturing, there have been recent developments in holistic synthetic biology that offer alternatives to existing strategies. Advances in comparative genomics and functional analysis of genome content provide the ability to generate “part lists” for engineering. Further, research is developing “blueprints” of metabolic reaction networks providing detailed information for improving biomanufacturing. In addition, stoichiometric and quantitative genome-scale metabolic modeling of multiple organisms is providing information not previously available. Finally, in many ways relating to all of these developments, the progress in biological applications of machine learning provides significant advantages for biomanufacturing optimization and design in general. These alternatives are free from one or more the limitations described above.

In particular, the development and expansion of computational biology has greatly advanced biomanufacturing. Metabolic modeling (MM) approaches are critical tools for identifying potential factors which may influence the production of a target biomolecule. Today, one of the most widely used and complete metabolic models available for use in driving metabolic engineering is the iML1515 genome-scale model of E. coli. Several methods focus on obtaining condition-specific models defined by omics datasets, which integrate the experimental data as additional constraints, thereby reducing the number of possible design variables. Besides obvious strengths, MM has certain limitations.

Secondary or alternative computational approaches may improve the optimization of biomanufacturing in certain technology areas. Unlike traditional metabolic modeling, which is based on mass and energy balances derived from reconstructed metabolic networks, data-driven algorithms, such as machine learning (ML) approaches, make predictions by extracting patterns from experimentally generated data.

ML-based computational strategies function by deriving patterns from data without the need for mechanistic understanding. However, such strategies require pathway analysis and MM to determine key engineering elements for ML-based combinatorics.

Despite these advances, there remains a need in the art for microorganism (e.g., bacterial) strains which are readily culturable and efficiently produce large amounts of a target biomolecule of interest, such as an amino acid.

### SUMMARY OF THE INVENTION

Provided herein are methods of engineering a target-biomolecule-producing bacterial cell. In some embodiments, a method of engineering a target-biomolecule-producing bacterial cell comprises (i) identifying a set of optimized parameters predicted to result in increased production of the target biomolecule by the bacterial cell; (ii) constructing a plurality of bacterial strains, each bacterial strain comprising one or more of the optimized parameters of the set of optimized parameters identified in (i); (iii) collecting target biomolecule production data from the strains constructed in (ii); (iv) performing a computational analysis of the data collected in (iii) in order to obtain a further optimized set of parameters that predict increased production of the target biomolecule; (v) repeating steps (ii), (iii), and (iv); and (vi) constructing one or more final bacterial strains, each bacterial strain comprising one or more of the optimized parameters of the set of optimized parameters identified in (i) or (iv). In some embodiments, the step of repeating is performed at least twice.

In some embodiments, a bacterial cell comprises a modified operon comprising a gene sequence encoding the target biomolecule. In some embodiments, a modified operon is operably linked to a non-native promoter. In some aspects, a method of engineering a target-biomolecule-producing bacterial cell comprises identifying a set of optimized parameters predicted to result in increased production of the target biomolecule by the bacterial cell, wherein the parameters are selected from the group consisting of host strain, inactivated genes, and overexpressed genes. In some embodiments, the parameters may further include (i) presence of endogenous operon comprising the gene sequence encoding the target biomolecule; (ii) chromosomal or plasmid localization of the modified operon; (iii) induction of the modified operon by Isopropyl β-D-1-thiogalactopyranoside (IPTG); (iv) growth time post-induction; and (v) culture medium type.

In some embodiments, a method of engineering a target-biomolecule-producing bacterial cell comprises performing a computational analysis. In some aspects, a computational analysis comprises machine learning (ML). In some aspects, a computational analysis further comprises metabolic modeling (MM). A method of engineering a target-biomolecule-producing bacterial cell may include collecting target biomolecule production data from constructed strains. In some embodiments, a step of collecting further comprises collecting one or more of bacterial cell growth rate data, sugar conversion data, and RNA-Seq data. In some embodiments, the bacterial cell is a bacterial cell of the strain ATCC 21277.

Provided herein are engineered bacterial cells capable of producing threonine, wherein the engineered bacterial cell comprises a chromosome comprising a metL deletion. An engineered bacterial cell may be an E. coli cell. In some embodiments, an E. coli cell is an E. coli cell of the strain ATCC 21277. In some embodiments, an engineered bacterial cell may include an attenuated metL gene. In some aspects, an engineered bacterial cell comprises a chromosome comprising a deletion of one or more of tdh, dapA, and dhaM. In some aspects, an engineered bacterial cell comprises a chromosome comprising a deletion of one or more of metL, tdh, dapA, and dhaM.

In some embodiments, an engineered bacterial cell may comprise a plasmid comprising a nucleotide sequence encoding a ppc gene. In some embodiments, an engineered bacterial cell may comprise a plasmid comprising a nucleotide sequence encoding an aspC gene. In some embodiments, an engineered bacterial cell may comprise a plasmid comprising a nucleotide sequence encoding a pntAB gene. In some aspects, an engineered bacterial cell may comprise one or more plasmids, each plasmid comprising one or more nucleotide sequences encoding one or more of a ppc gene, an aspC gene, and a pntAB gene. In some aspects, an engineered bacterial cell may comprise a chromosome comprising at least two copies of one or more genes selected from ppc, aspC, and pntAB, thereby promoting overexpression of the one or more genes.

In some embodiments, an engineered bacterial cell may include a chromosome comprising one or more of: (i) a ppc gene operably linked to a non-native promoter; (ii) an aspC gene operably linked to a non-native promoter; and (iii) a pntAB gene operably linked to a non-native promoter. A non-native promoter may be a tac promoter.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows a general scheme representing an embodiment of the methods described herein for the engineering of threonine-producing bacterial cells.

FIG. 2 shows a fragment of metabolic map of E. coli representing threonine biosynthesis and adjacent pathways, derived from (King, Z. A., et al., Escher: A Web Application for Building, Sharing, and Embedding Data-Rich Visualizations of Biological Pathways, PLoS Comput Biol, 2015. 11(8); p. e1004321). Selected genes are highlighted as follows: (green) carbon backbone; (blue) threonine biosynthesis; (pink) competing pathways, (yellow) threonine catabolism; (orange) threonine transport, and (red) other.

FIG. 3 shows a plot of actual threonine production values (x-axis) vs. predicted values (y-axis) in experiments testing the ability of AI to predict threonine production levels. The training set (2444 samples) is shown in blue, and the testing set (405) samples in red, with the testing set following the trend of the training set.

FIG. 4 shows a plot of actual threonine production values (x-axis) vs. predicted values (y-axis) for a model trained on samples with threonine production levels less than or equal to 1.2 g/L. The training set is shown in blue, and the testing set in red. Red dots to the right of 1.2 (vertical red line) show how the model performs outside its training range. Dots above 0.8 (horizontal red line) represent the high prediction range for this model.

FIGS. 5A-5B show representations of threonine production values as a function of construction variables, (knockouts and overexpressed genes). The samples were shown in 2 groups: 7asdO (FIG. 5A) and 7asdT (FIG. 5B) (naming explained in Table 2, infra). Displayed samples were collected at 24 hours, and threonine operon transcription was induced by IPTG.

FIG. 6 shows a plot of actual threonine production values (x-axis) vs. predicted values (y-axis) in experiments testing the ability of AI to predict threonine production levels after a third round of AI-driven strain design. The training set (3849 samples) is shown in blue, and the testing set (481 samples) in red, with the testing set following the trend of the training set.

FIG. 7 shows RNA expression data showing conserved patterns of expression changes in strains producing increased amounts of threonine. Two differently behaving groups represent strains constructed in the project, and strains constructed in prior studies where extensive selection component was a part of the engineering. Effects on lysC and rhtA expression levels are highlighted.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### DEFINITIONS

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±20% of that numerical value, such as less than or equal to ±10%, less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5% less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, “optional” or “optionally” means that the subsequently described event or circumstance can or cannot occur, and that the description includes instances where the event or circumstance occurs and instances where it does not.

As used herein, the term “yield” refers to the amount of a product produced in relation to the amount of a starting material. With respect to amino acids produced by a microorganism, yield refers to the amount of amino acid produced with respect to the amount of intermediate, precursor or nutrient provided. For example, when 100 grams of dextrose is supplied to a microorganism which produces 25 grams of L-isoleucine, the yield of L-isoleucine, with respect to the dextrose, is 25%.

As used herein, the term “strain” refers to bacteria of a particular species which have common characteristics. Unless indicated to the contrary, the terms “strain” and “cell” are used interchangeably herein. As one skilled in the art would recognize, bacterial strains are composed of individual bacterial cells. Further, individual bacterial cells have specific characteristics (e.g., a particular growth rate or level of target biomolecule production) which identifies them as being members of their particular strain.

As used herein, the term “mutation” refers to an insertion, deletion or substitution in a nucleic acid molecule. When present in the coding region of a nucleic acid, a mutation may be “silent” (i.e., results in no phenotypic effect) or may alter the function of the expression product of the coding region. When a mutation occurs to the regulatory region of a gene or operon, the mutation may either have no effect or alter the expression characteristics of the regulated nucleic acid.

As used herein, the term “modulation of expression” may refer to up-regulation of expression by, for example, a regulated or constitutive promoter inserted upstream of a gene, or by gene cloning in a multi-copy plasmid. The term “modulation of expression” may refer to down-regulation of expression by, for example, replacing a native promoter with a “weaker” promoter, or by a complete gene inactivation.

As used herein, the term “biomolecule” or “biological molecule” refers to a any of numerous substances produced by cells and living organisms. Biomolecules have a wide range of sizes and structures, and may perform a vast array of functions. Non-limiting examples of biomolecules include saccharides (e.g., monosaccharides, disaccharides, etc.), carbohydrates, fatty acids, lipids (e.g., glycolipids, phospholipids, sterols, etc.), nucleosides, nucleotides, nucleic acids (e.g., deoxyribonucleic acids (DNA), ribonucleic acids (RNA)), amino acids, polypeptides, proteins, vitamins, neurotransmitters, metabolites, and enzymes. Biomolecules may be endogenous, synthetic, or modified.

As used herein, the term “phenotype” refers to observable physical characteristics dependent upon the genetic constitution of a microorganism. Examples of phenotypes include the ability to express particular gene products and the ability to produce certain amounts of a particular amino acid in a specified amount of time.

As used herein, the term “over-produce” refers to the production of a biomolecule by a cell in an amount greater than the amount produced by a reference strain (e.g., a parent strain). One example of an over-producing strain would be a strain generated from a parent strain (i.e., the reference strain) using mutagenesis which produces more of a particular target biomolecule (e.g., a particular amino acid, such as threonine) than the parent. Thus, the strain generated by mutagenesis would “over-produce” the target biomolecule in comparison to the parent, reference strain.

As used herein, the term “attenuate” means to reduce the function of. For example, as used herein, an “attenuated gene” is a gene whose expression and/or function is reduced relative to that of a non-attenuated version of the gene. An attenuated gene is a gene may have an activity level that is less than 100% (e.g., 99%, 95%, 90%, 80%, 50%, 25%, 20%, 10%, 5%, or 0%) of the activity level of a non-attenuated version of the gene.

As used herein, the term “operon” refers to a unit of bacterial gene expression and regulation. Operons are generally composed of regulatory elements and at least one open reading frame (ORF). An example of an operon is the threonine operon of E. coli which is composed of a regulatory region and three open reading frames. Another example of an operon is the isoleucine operon of E. coli which is composed of a regulatory region and four open reading frames.

As used herein, the term “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

As used herein, the term “operably linked” means in this context the sequential arrangement of the promoter polynucleotide according to the disclosure with a further oligo- or polynucleotide, resulting in transcription of said further polynucleotide.

As used herein, the term “parent strain” refers to a strain of a microorganism subjected to mutagenesis to generate a microorganism of the invention. Thus, use of the phrase “parent strain” does not necessarily equate with the phrase “wild-type” or provide information about the history of the referred to strain.

As used herein, the terms “engineered bacterial cell” refers to a modified bacterial cell, such as E. coli, wherein the modification can be selected from e.g., enhanced expression of a gene, inhibited expression of a gene, introduction of new gene(s), introduction of mutant gene(s), or mutation of gene(s), wherein the enhanced expression or inhibited expression of a gene can be achieved by using common techniques in the art, such as gene deletion, changed gene copy number, introduction of a plasmid, changed gene promoter (e.g. by using a strong of weak promoter) etc. In some embodiments, an engineered bacterial cell is a modified bacterial cell capable of producing high levels of a biomolecule, such as amino acid.

The terms “genetically modified host cell,” “recombinant host cell,” and “recombinant strain” are used interchangeably herein and refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally occurring organism from which it was derived. It is understood that in some embodiments, the terms refer not only to the particular recombinant host cell in question, but also to the progeny or potential progeny of such a host cell.

As used herein, the term “modification profile” refers to one or more factors altered in a strain with the goal of optimizing the production of a biomolecule, such as an amino acid. Such factors may be, without limitation, gene deletion, gene overexpression (e.g., by cloning vectors or promoter modifications), host strain, status of original/endogenous operon, modification of operon, copy number for modified operon, presence (status) of a particular gene in the bacterial chromosome, operon induction time point, growth stage, and culture medium.

### Methods of Engineering Bacterial Strains to Produce a Target Biomolecule

The present disclosure provides methods for “agnostic” bacterial strain engineering. Methods described herein may be applied to, for example, optimization of production of a target biomolecule in bacterial cells, such as E. coli. In some embodiments, methods of bacterial strain engineering may comprise the following steps: (1) perform a pathway analysis; use general known gene pathway information to select genes whose inactivation or over-expression may increase production of a target biomolecule; (2) construct a set of bacterial strains comprising one or more over-expressed or inactivated genes; (3) collect target biomolecule production data from the strains constructed in (2); (4) perform a computational analysis to generate new potential strains comprising combinations of two or more genetic modifications predicted to promote increased target biomolecule production; (5) find additional new genes affecting target biomolecule production from metabolic modeling and ML analysis of expression data; and (6) construct and test a further set of engineered bacterial strains designs guided by artificial intelligence (AI).

In some embodiments, methods of bacterial strain engineering may comprise an algorithm. In some embodiments, an algorithm represents a sequence of prototyped processes. In some embodiments, a method of bacterial strain engineering may comprise a process comprising: (1) selecting genetic elements for strain engineering through Metabolic Modeling (MM), and identifying impactful genes as initial targets for strain engineering efforts; (2) designing engineered bacterial strains by selecting nucleotide sequences from one or more comparative genomics databases, choosing promoters (e.g., native or non-native promoters) to up or down-regulate selected genes from baseline gene expression values, and designing primers and recombinant molecules for strain construction; (3) constructing a plurality of bacterial strains based on MM-guided designs obtained in (2) and their testing in Automated Lab by multiplex strain engineering, culture growth and production testing, and RNA-SEQ data collection; (4) performing a computational analysis using artificial intelligence (AI) data analysis comprising executing AI models from HTB data (Deep Learning for production data, Random Forest for RNA-SEQ), and MM filtering; (5) constructing and testing AI-designed improved strains based on the computational analysis of (4); and (6) iterating steps 4 and 5.

In some embodiments, the present disclosure teaches methods of predicting the effects of particular genetic alterations being incorporated into a given host strain. In some aspects, the disclosure provides methods for generating proposed genetic alterations that should be incorporated into a given host strain, in order for said host to possess a particular phenotypic trait or strain parameter. In some aspects, the present disclosure provides methods for using predictive models and/or computational algorithms to design novel bacterial strains with a desired phenotype. In some embodiments, provided herein are methods for using predictive models and/or computational algorithms to design novel bacterial strains that are capable of producing a target biomolecule.

In some embodiments, the present disclosure provides methods for generating proposed genetic alterations that should be incorporated into a given host strain, in order for said host to possess an ability to overproduce a target biomolecule.

In some embodiments, the present disclosure teaches a system which generates proposed genetic modifications to host strains based on previous experimental data. In some embodiments, the recommendations of the present system are based on the results from the immediately preceding screening. In other embodiments, the recommendations of the present system are based on the cumulative results of one or more (e.g., one, two, three, or more) of the preceding screenings.

In some embodiments, the recommendations of the present system are based on scientific insights. For example, in some embodiments, the recommendations are based on known properties of genes (from sources such as annotated gene databases and the relevant scientific literature), codon optimization, or other hypothesis driven sequence and host optimizations.

In some embodiments, the proposed genetic modifications to a host strain recommended by systems or predictive models described herein are carried out by the utilization of molecular tools known in the art.

Provided herein are methods of engineering a target-biomolecule-producing bacterial cell. In some embodiments, a method of engineering a target-biomolecule-producing bacterial cell comprises: (i) identifying a set of optimized parameters predicted to result in increased production of the target biomolecule by the bacterial cell; (ii) constructing a plurality of bacterial strains, each bacterial strain comprising one or more of the optimized parameters of the set of optimized parameters identified in (i); (iii) collecting target biomolecule production data from the strains constructed in (ii); (iv) performing a computational analysis of the data collected in (iii) in order to obtain a further optimized set of parameters that predict increased production of the target biomolecule; (v) repeating steps (ii), (ii), and (iv); and (vi) constructing one or more final bacterial strains, each bacterial strain comprising one or more of the optimized parameters of the set of optimized parameters identified in (v), thereby engineering a target-biomolecule-producing bacterial cell.

In some embodiments, a method of engineering a target-biomolecule-producing bacterial cell comprises identifying a set of optimized parameters predicted to result in increased production of a target biomolecule by a bacterial cell. In some embodiments, parameters may include intrinsic parameters (e.g., genome alterations) and/or extrinsic parameters (e.g., growth conditions). Non-limiting examples of parameters include background host strain, inactivated genes, overexpressed genes, presence of endogenous operon comprising a copy of a gene sequence encoding the target biomolecule, chromosomal or plasmid localization of a modified operon comprising a copy of a gene sequence encoding the target biomolecule, induction of transcription of an operon comprising a copy of a gene sequence encoding the target biomolecule by an inducing agent (e.g., Isopropyl β-d-1-thiogalactopyranoside (IPTG)), growth time post-induction, total growth time, and culture medium. In some embodiments, optimized parameters may be selected based on existing data. For example, selecting genes for overexpression or inactivation for use as a parameter in a method of engineering a biomolecule-producing bacterial cell may be accomplished using published or otherwise publicly available datasets.

In some embodiments, a method of engineering a target-biomolecule-producing bacterial cell comprises constructing a plurality of bacterial strains, each bacterial strain comprising one or more optimized parameters of a set of optimized parameters. In some embodiments, optimized parameters may have been identified in a prior step of such a method. Bacterial strains may be constructed using tools and techniques known in the art, and/or as discussed supra.

In some embodiments, a method of engineering a target-biomolecule-producing bacterial cell comprises phenotypically characterizing initial constructed strains. In some embodiments, characterizing initial constructed strains may involve one or more of collecting target biomolecule production data and collecting RNA-Seq data from constructed strains. For example, the ability of a constructed strain to produce a target biomolecule may be quantified using tools and techniques known in the art, and as discussed in the Examples provided herein. RNA-seq data may be obtained from constructed strains using tools and techniques known in the art, and as discussed in the Examples provided herein. Other types of production data that may be obtained from constructed strains include, but are not limited to, sugar (e.g., glucose) conversion and bacterial cell culture growth rate Experimental data obtained from constructed strains may be used to inform the selection of and/or optimization of parameters for one or more subsequent rounds of strain construction. Experimental data obtained from constructed strains may be used as input parameters for subsequent data analyses (such as, for example, computational analyses), which may be used to inform the selection of and/or optimization of parameters for one or more subsequent rounds of strain construction.

Methods of engineering a target-biomolecule-producing bacterial cells or bacterial strains may comprise computational approaches. Computational approaches may include, without limitation, machine learning (ML) approaches, metabolic modeling (MM), and RNA-Seq analyses. In some embodiments, computational analyses may be used to guide engineering of novel target-biomolecule-producing bacterial strains.

MM approaches to bacterial strain engineering are thoroughly described in Wang, Dash, et al. (2017) A review of computational tools for design and reconstruction of metabolic pathways.” Synth Syst Biotechnol 2(4); 243-252, which is incorporated herein by reference in its entirety. MM approaches can guide organism engineering by providing a “wiring diagram” for metabolic pathways effecting production of a target biomolecule, such as, for example, an amino acid. However, MM approaches typically do not include or account for various forces around the so-called “wiring diagram”, including, without limitation, kinetic parameters, impacts of regulatory factors, and complex effects such as product tolerance, cell stresses, and genetic stability (Oyetunde et al. (2018). Leveraging knowledge engineering and machine learning for microbial bio-manufacturing. Biotechnol Adv. 36(4); 1308-1315). Artificial intelligence (AI) strategies derive patterns from data without the need for a mechanistic understanding of the data. However, AI strategies require a set of initial conditions, such as by pathway analyses and MM to determine key engineering elements for AI combinatories.

Besides obvious strengths, MM has certain limitations. For example, underlying stoichiometric matrices are often incomplete, and may ignore non-metabolic factors. Machine learning (ML) approaches may address certain knowledge gaps, such as those described above.

Data-driven algorithms can make predictions by extracting patterns from, inter alia, experimentally generated data. A general approach to the automated Design, Build, Test, and Learn (DBTL) process guided by ML is described in Carbonell, Le Feuvre, et al. (2020) In silico design and automated learning to boost next-generation smart biomanufacturing. Synth Biol (Oxf) 5(1); and Ramzi, Baharum, et al. (2020) Streamlining Natural Products Biomanufacturing With Omics and Machine Learning Driven Microbial Engineering Front Bioeng Biotechnol 8: 608918, each of which is incorporated herein by reference in its entirety. The application of ML methods for enzyme engineering is demonstrated in Siedhoff, Schwaneberg, & Davari (2020) Machine learning-assisted enzyme engineering. Methods Enzymol 643: 281-315, which is incorporated by reference herein in its entirety.

In some embodiments, multiple rounds of parameter optimization may be employed in order to generate bacterial strains that produce a target biomolecule at high levels. For example, steps of strain construction followed by steps of data (e.g., target biomolecule production data, RNA-Seq data, sugar conversion data, and/or bacterial cell culture growth data) collection may be repeated one, two three, four, five, or six or more times. One of skill in the art will be aware of the advantages and benefits to multiple rounds of data generation.

In some embodiments, methods of engineering a target-biomolecule-producing bacterial cell are useful for the identification and production of bacterial strains capable of producing substantial quantities of a target biomolecule when grown in culture. In some embodiments, when grown in culture, strains produced by the methods provided herein include strains which are capable of producing at least about 2 g/L to about 10 or more g/L of a target biomolecule in about 24 hours. In particular, when grown in culture, strains produced by the methods provided herein include strains which are capable of producing at least about 2 g/L, at least about 3 g/L, at least about 4 g/L, at least about 5 g/L, at least about 6 g/L, at least about 7 g/L, at least about 8 g/L, at least about 9 g/L, at least about 10 or more g/L of a target biomolecule in about 24 hours. In some embodiments, strains produced by the methods provided herein are capable of producing at least 2.0 g/L, at least 3.0 g/L, at least 4.0 g/L, at least 5.0 g/L, at least 6.0 g/L, at least 7.0 g/L, at least 8.0 g/L, at least 9.0 g/L, or at least 10.0 or more g/L of a target biomolecule by about 24 hours of growth in culture. In some preferred embodiments, methods of engineering a target-biomolecule-producing bacterial cell are useful for the identification and production of bacterial strains capable of producing at least 8.0 g/L of a target biomolecule by about 24 hours of growth in culture.

### Engineered Threonine-Producing Bacterial Strains

Threonine is an essential amino acid that has found use in feed stock, pharmaceuticals, and dietary supplements. In the 1970s, strains of Corynebacterium (Brevibacterium) and E. coli were engineered to produce threonine in industrial quantities (Kase and Nakayama, 1972) (Hirakawa, Tanaka et al. 1973). Microorganism-based biomanufacturing of threonine has seen gradual improvements (see, e.g., Wittmann and Becker 2007) since its inception, driving costs down. Biomanufacturing has become the the economically viable pathway replacing traditional bulk chemical production. (See, e.g., Clomburg, Crumbley et al. 2017).

In some embodiments, novel bacterial strains of the present invention have the following characteristics:
1) they contain at least one threonine (thr) operon (i.e., contain at least one set of the genes encoding biosynthetic enzymes) which (a) is integrated into the bacterial chromosome or is present in an extrachromosomal element (e.g., a plasmid), and (b) is under the control of a non-native promoter; and
2) they are capable of producing threonine at levels that are substantially higher than those achieved by wild-type strains upon growth in culture.

In some embodiments, the invention relates to an engineered bacterial cell. For example, in some embodiments, the invention relates to an engineered E. coli cell.

The threonine (thr) operon on the chromosome of cells of bacterial strains included within the scope of the invention encodes enzymes necessary for threonine biosynthesis. Due to the fact that several enzymes are capable of catalyzing reactions to produce various intermediates in the threonine pathway, the genes present in the threonine operon employed can vary. For example, the threonine operon can be composed of an aspartate kinase I-homoserine dehydrogenase I (AK-HD I) gene (thrA), a homoserine kinase gene (thrB), and a threonine synthase gene (thrC). Suitable threonine operons may be obtained, for example, from E. coli strains deposited with the American Type Culture Collection (ATCC), 10801 University Blvd., Manassas, Va. 20110-2209, USA and assigned ATCC Deposit Nos. 21277.

In some embodiments, multiple copies of the thr operon may be present on the chromosomes of bacterial cells of the invention. In some embodiments, multiple copies of the thr operon may be present in the bacterial cells, whether integrated into the bacterial chromosome or supplied on a plasmid. Increased copy number of the thr operon will generally result in increased expression of the genes of this operon upon induction.

In some embodiments, the thr operon contains at least one non-attenuated gene (i.e., expression of the gene is not suppressed by the levels (extra- and/or intra-cellular) of one or more of the threonine biosynthetic enzymes and/or the products thereof (e.g., threonine). In some embodiments, strains may also contain a thr operon having a defective thr attenuator (the regulatory region downstream of the transcription initiation site and upstream of the first structural gene) or a thr operon that lacks the thr attenuator altogether. For example, a strain may contain a modified thrA gene which lacks a leader peptide.

In some embodiments, the thr operon encodes one or more feedback-resistant threonine biosynthetic enzymes (e.g., the activity of the enzyme is not inhibited by the extra- and/or intra-cellular levels of the intermediates and/or products of threonine biosynthesis). For example, in some embodiments, the thr operon may contain a gene that encodes a feedback-resistant AK-HD, such as a feedback-resistant AK I-ID I. Use of a feedback-resistant AK-HD provides a higher level of enzymatic activity for threonine biosynthesis, even in the presence of the threonine being produced (See U.S. Pat. No. 7,767,431 B2).

Expression of the threonine operon(s) in strains of the present disclosure will generally be controlled by a non-native promoter (i.e., a promoter that does not control expression of the thr operon in bacterial strains normally found in nature (Le., wild-type strains). Replacing the native promoter of the threonine biosynthetic enzymes with a strong non-native promoter to control expression of the thr operon results in higher threonine production even with only a single, genomic copy of the thr operon. Non-limiting examples of promoters suitable for use in E. coli include: the lac promoter, the trp promoter, the Pz promoter of λ bacteriophage, the P_{R} promoter, the Ipp promoter, and the tac promoter. In some preferred embodiments, a non-native promoter used to control expression of the thr operon in an engineered bacterial cell is a tac promoter.

In addition to the threonine operon, cells of the inventive bacterial strains may also contains at least one gene encoding aspartate semialdehyde dehydrogenase (asd) either integrated into their chromosomes or present on an extrachromosomal element (e.g., a plasmid). For example, the chromosome in cells of the present invention may contain at least one asd gene, at least one thrA gene, at least one thrB gene and/or at least one thrC gene. Of course, one, two, three, or more copies of each of these genes may be present.

Engineered bacterial cells of the present invention may comprise at least one threonine operon. In some embodiments, engineered bacterial cells of the present invention may comprise at least one threonine operon comprising at least one variation relative to a wild-type threonine operon. In some embodiments, a threonine operon of an engineered bacterial cell comprises the wild type sequence of the thrABC operon and a wild-type asd gene. In some embodiments, a threonine operon of an engineered bacterial cell comprises the wild type sequence of the thrABC operon and a mutated asd gene. A mutated asd gene may comprise one or more amino acid changes relative to wild-type asd. In some embodiments, a threonine operon of an engineered bacterial cell comprises a feedback-resistant thrA gene. A feedback-resistant thrA gene may comprise one or more amino acid changes relative to wild-type thrA. In some embodiments, a threonine operon of an engineered bacterial cell comprises two copies of the lacIq gene, which may increase the growth rate of plasmid-carrying strains. In some embodiments, the endogenous threonine operon of an engineered bacterial cell may be deleted or otherwise non-functional. In some embodiments, the endogenous asd of an engineered bacterial cell may be deleted or otherwise non-functional. In some embodiments, the endogenous threonine operon of an engineered bacterial cell may be unmodified. In some embodiments, a threonine operon of an engineered bacterial cell may be under the control of a tac promoter. In some embodiments, a threonine operon of an engineered bacterial cell may be under the control of a tac promoter, and said threonine operon may comprise an asd gene. In some embodiments, a threonine operon of an engineered bacterial cell may be under the control of a tac promoter, and said threonine operon may not comprise an asd gene. In some embodiments, a threonine operon of an engineered bacterial cell may be under the control of a tac promoter, and said threonine operon may comprise a feedback-resistant thrA gene. In some embodiments, a threonine operon of an engineered bacterial cell may be under the control of a tac promoter, and said threonine operon may comprise an asd gene and a feedback-resistant thrA gene. In some embodiments, a threonine operon of an engineered bacterial cell may be under the control of a tac promoter, and said threonine operon may comprise an asd gene, a feedback-resistant thrA gene, and at last two copies of a lacIq gene. In some embodiments, an engineered bacterial cell may comprise at least one copy of a lacIq gene provided on a plasmid. In some embodiments, an engineered bacterial cell may comprise at least two copies of a lacIq gene provided on a plasmid.

Threonine production by engineered bacterial cells of the present disclosure may be chemically induced. Engineered bacterial cells may comprise an activatable or inducible promoter allowing for control over the expression of gene(s) downstream of said promoter. For example, in some embodiments, an engineered bacterial cell may comprise a promoter that is chemically inducible, such as, by Isopropyl β-d-1-thiogalactopyranoside (IPTG). In some embodiments, threonine production may be induced by treating with IPTG an engineered bacterial cell comprising an IPTG-inducible promoter upstream of a threonine operon.

An engineered E. coli cell may comprise a genome of any background host strain. In some embodiments, an engineered E. coli cell of the present disclosure may comprise a genome of a particular background host strain of E. coli. Non-limiting examples of E. coli background host strains include K-12 (MG1655), ATCC21278 (Shiio 1971), ATCC21277 ((Shiio 1971), NRRL B-21593 (Wang 1999), NRRL B-30319 (Liaw 2007), NRRL B-30823(8) (D'Elia 2010), NRRL B-30316 (Liaw 2007), NRRL B-30317 (Liaw 2007), and NRRL B-30318 (Liaw 2007). In some embodiments, an engineered bacterial cell of the present disclosure may be of a MG1655 host strain. In some embodiments, an engineered bacterial cell of the present disclosure may be of an ATCC 21277 host strain. In some embodiments, an engineered bacterial cell of the present disclosure may be of an ATCC21278 host strain, an ATCC21277 host strain, a NRRL B-21593 host strain, a NRRL B-30319 host strain, a NRRL B-30823(8) host strain, a NRRL B-30316 host strain, a NRRL B-30317 host strain, or a NRRL B-30318 host strain. In some preferred embodiments of the invention, an engineered E. coli cell may be of an MG1655 host strain or of an ATCC21277 host strain. In some preferred embodiments of the invention, an engineered E. coli cell may be of an ATCC 21277 host strain.

Engineered bacterial strains of the present disclosure may be constructed combinatorially. For example, a bacterial cell from an engineered bacterial strain may comprise one or more genotypic or phenotypic alterations resulting in an enhanced ability to produce threonine. Such alterations may be a combination of gene deletions and gene overexpression. Gene overexpression may be achieved, without limitation, by an increase in the copy number of a gene of interest, e.g., by the presence of a gene of interest on an extrachromosomal element (e.g., a plasmid). Engineered bacterial strains of the present invention may represent, without limitation, combinations of: (1) individual or combined deletions of one or more (e.g., one, two, or three or more) E. coli genes; (2) one or more background host strains, (3) one more modifications of a thr operon present on the bacterial chromosome or on an extrachromosomal element, for example, as discussed above; and (4) one or more overexpressed genes.

In some embodiments, an engineered bacterial strain may comprise a genome comprising one or more deletions of one or more genes selected from the group consisting of dapA. dhaM, lysA, lysC, metL, ptsG, rhtA, and tdh. In some embodiments, an engineered bacterial strain may comprise overexpression of one or more genes selected from the group consisting of aceBA, aspC, pntAB, ppc, pyc, rhtA and zwf. In some embodiments, a gene may be overexpressed due to an increase in copy number of a gene in a bacterial cell, preferably through delivery of said gene to said bacterial cell on an extrachromosomal element (e.g., plasmid). In some embodiments, an engineered bacterial strain may comprise one or more of (1) a genome comprising one or more deletions of one or more genes selected from the group consisting of dapA, dhaM, lysA, lysC, metL, ptsG, rhtA, and tdh; and (2) overexpression of one or more genes selected from the group consisting of aceBA, aspC, pntAB, ppc, pyc, rhtA, and zwf.

In some embodiments, an engineered bacterial strain may comprise a genome comprising one or more deletions of one or more genes selected from the group consisting of dapA, dhaM, lysA, lysC, metL, ptsG, rhtA, and tdh. In some embodiments, an engineered bacterial strain may comprise overexpression of one or more genes selected from the group consisting of aceBA, aspC, pntAB, ppc, pyc, rhtA, and zwf. In some embodiments, a gene may be overexpressed due to an increase in copy number of a gene in a bacterial cell, preferably through delivery of said gene to said bacterial cell on an extrachromosomal element (e.g., plasmid). In some embodiments, an engineered bacterial strain may comprise one or more of (1) a genome comprising one or more deletions of one or more genes selected from the group consisting of dapA, dhaM, lysA, lysC, metL, ptsG, rhtA, and tdh; and (2) overexpression of one or more genes selected from the group consisting of aceBA, aspC, pntAB, ppc, pyc, rhtA, and zwf.

Engineered bacterial strains of the present disclosure may be prepared by any of the methods and techniques known and available to those skilled in the art. Illustrative examples of suitable methods for constructing the inventive bacterial strains include gene integration techniques (e.g., mediated by transforming linear DNA fragments and homologous recombination) and transduction mediated by the bacteriophage P1. These methods are well known in the art and are described, for example, in J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); M. Singer and P. Berg, Genes & Genomes, University Science Books, Mill Valley, Calif. (1991); J. Sam-brook, E. F. Fritsch and T. Maniatis, Molecular Cloning: A Laboratory Manual, 2d ed., Cold Spring Harbor Laboratory Press, Cold Spring Harbor, N.Y. (1989); P. B. Kaufman et al., Handbook of Molecular and Cellular Methods in Biology and Medicine, CRC Press, Boca Raton, Fla. (1995); Methods in Plant Molecular Biology and Biotechnology, B. R. Glick and J. E. Thompson, eds., CRC Press, Boca Raton, Fla. (1993); and P. F. Smith-Keary, Molecular Genetics of Escherichia coli, The Guilford Press, New York, N.Y. (1989), the entire disclosure of each of which is incorporated herein by reference.

Bacterial strains of the present invention include strains which are capable of producing substantial quantities of threonine when grown in culture. In some embodiments, when grown in culture, strains of the invention include strains which are capable of producing at least about 2 g/L to about 10 or more g/L of threonine in about 24 hours. In particular, when grown in culture, strains of the invention include strains which are capable of producing at least about 2 g/L, at least about 3 g/L, at least about 4 g/L, at least about 5 g/L, at least about 6 g/L, at least about 7 g/L, at least about 8 g/L, at least about 9 g/L, at least about 10 or more g/L of threonine in about 24 hours. In some embodiments, an engineered bacterial cell of the present disclosure is an engineered bacterial cell capable of producing at least 2.0 g/L, at least 3.0 g/L, at least 4.0 g/L, at least 5.0 g/L, at least 6.0 g/L, at least 7.0 g/L, at least 8.0 g/L, at least 9.0 g/L, or at least 10.0 or more g/L of threonine by about 24 hours of growth in culture.

In some embodiments, bacterial strains include strains which are capable of producing threonine at a rate of at least about 0.08 g/L/hr, at least about 0.4 g/L/hr.

As discussed above, engineered bacterial strains may generally be altered in at least one gene related to production of a particular amino acid (e.g., threonine) as compared to wild-type strains. As also discussed above, bacterial strains of the invention which over-produce an amino acid (e.g., threonine) include strains which contain at least one threonine operon which (a) is integrated into the bacterial chromosome the chromosome or is present on an extrachromosomal element (e.g., a plasmid); and (b) is under control of a non-native promoter. In some embodiments, strains may contain phenotypic changes related to one or more of the following: (1) the elimination or reduction of feed-back control mechanisms for one, two, three or more biosynthetic pathways which lead to production of amino acids (e.g., threonine); (2) the enhancement of metabolic flow by either increasing expression of genes which encode rate-limiting enzymes of biosynthetic pathways which lead to the production of amino acids (e.g., threonine) or precursors thereof (e.g., Aspartate); (3) the inhibition of degradation pathways involving either the desired amino acid end product (e.g., L-threonine or L-isoleucine), intermediates (e.g., homoserine), and/ or precursors (e.g., aspartate); (4) increased production of intermediates and/or and precursors; (5) when the pathway which leads to production of a desired amino acid end product is branched, inhibition of branches which do not lead the desired end product or an intermediate and/or a precursor of the desired end product (e.g., inhibiting the E. coli methionine pathway, when the desired end product is threonine); (6) inhibition of competing biosynthetic pathways; (7) alterations in membrane permeability to optimize uptake of energy molecules (e.g., glucose), intermediates and/or precursors; (8) alterations in membrane permeability to optimize amino acid end product (e.g., threonine) excretion; (9) the enhancement of growth tolerance to relatively high concentrations of end products (e.g., amino acids, e.g., threonine), metabolic waste products (e.g., acetic acid), or metabolic side products (e.g., amino acid derivatives) which are inhibitory to bacterial cell growth; (10) the enhancement of resistance to high osmotic pressure during cultivation resulting from increased concentrations of carbon sources (e.g., glucose) or end products (e.g., amino acids); (11) the enhancement of growth tolerance to changes in environmental conditions (e.g., pressure, temperature, pH, etc.); and (12) increasing activities of enzymes involved in the uptake and use of carbon sources in the culture medium. In some particular embodiments, engineered bacterial strains may generally be altered in at least one of the following phenotypic traits or functional categories: (1) carbon backbone; (2) threonine biosynthesis; (3) threonine catabolism; and (4) threonine transport; (5) alteration of competing pathways; and (6) other functional categories.

In some embodiments, engineered bacterial strains may comprise one or more deletions relative to wild-type strains. In some embodiments, engineered bacterial strains may comprise combinations of deleted genes. In some embodiments, engineered bacterial strains may comprise combinations of overexpressed genes. In some embodiments, engineered bacterial strains may comprise combinations of deleted and overexpressed genes. Overexpressed genes may be “supplemental” overexpressed genes delivered to the engineered bacterial strain on a plasmid. Genes with altered expression profiles (e.g., deleted genes and/or overexpressed genes) in an engineered bacterial strain of the present disclosure may represent one or more functional categories. For example, genes with altered expression profiles of engineered bacterial strains may represent one or more of: (1) carbon backbone; (2) threonine biosynthesis; (3) threonine catabolism; (4) threonine transport; (5) alteration of competing pathways; and (6) other functional categories. Non-limiting examples of genes associated with carbon backbone include pyc, zwf, aceBA, ptsG, dhaM, and ppc. Non-limiting examples of genes associated with threonine biosynthesis include: thrABC, asd, lysC, aspC, and metL. Non-limiting examples of genes associated with threonine transport include: rhtA. Non-limiting examples of genes associated with alteration of competing pathways include aspC, lysA, and dapA. For example, manipulation of the expression of lysA may be expected to increase the amount of amino acid precursors available for threonine production (Lee, Park et al. 2007). Non-limiting examples of other genes with the potential to impact threonine production in an engineered bacterial cell include pntAB.

In some embodiments, provided herein is an engineered bacterial cell capable of producing threonine, wherein the engineered bacterial cell comprises a chromosome comprising a metL deletion. metL is homologous to the thrA gene, and encodes bifunctional aspartokinase/homoserine dehydrogenase 2. The engineered bacterial cell may be an E. coli cell.

In some embodiments, provided herein is an engineered bacterial cell capable of producing threonine, wherein the engineered bacterial cell comprises a chromosome comprising an attenuated metL gene. Non-limiting examples of attenuated genes include those under the control of a promoter that is weaker than the native promoter that normally controls expression of the gene, those that comprise a mutation that limits but does not abolish the enzymatic function of the protein encoded by the gene, and those that otherwise exhibit reduced expression or activity relative to a non-attenuated version of the gene. In some embodiments, an engineered bacterial cell may comprise a chromosome comprising a deletion of one or more of tdh, dapA, and dhaM. In some embodiments, an engineered bacterial cell may comprise a chromosome comprising a deletion of one or more of tdh, dapA, and dhaM, in addition to a deletion of metL. In some embodiments, an engineered bacterial cell comprises a chromosome comprising a deletion of one or more of metL, tdh, dapA, and dhaM. In some embodiments, an engineered bacterial cell comprises a chromosome comprising one or more of an attenuated metL gene, an attenuated tdh gene, an attenuated dapA gene, and an attenuated dhaM gene.

In some embodiments, an engineered bacterial cell may comprise a plasmid. In some embodiments, an engineered bacterial cell may comprise a plasmid comprising a nucleotide sequence encoding one or more of a ppc gene, an aspC gene, and a pntAB gene. In some embodiments, an engineered bacterial cell may comprise a plasmid comprising a nucleotide sequence encoding one or more of a ppc gene. In some embodiments, an engineered bacterial cell may comprise a plasmid comprising a nucleotide sequence encoding an aspC gene. In some embodiments, an engineered bacterial cell may comprise a plasmid comprising a nucleotide sequence encoding a pntAB gene. In some embodiments, an engineered bacterial cell comprises a chromosome comprising at least two copies of one or more genes selected from ppc, aspC, and pntAB, thereby promoting overexpression of the one or more genes. One or more of the at least two copies of the one or more genes may be operably linked to a non-native promoter, such as, for example, a tac promoter. In some embodiments, an engineered bacterial cell comprises a chromosome comprising one or more of: (i) a ppc gene operably linked to a non-native promoter; (ii) an aspC gene operably linked to a non-native promoter, and (iii) a pntAB gene operably linked to a non-native promoter. In some embodiments, a non-native promoter may be a tac promoter.

In some embodiments, an engineered bacterial cell may comprise (i) a chromosome comprising deletions of one or more genes selected from the group consisting of metL, tdh, dapA, and dhaM; and (ii) one or more plasmids, each plasmid comprising one or more nucleotide sequences encoding one or more of a ppc gene, an aspC gene, and a pntAB gene. In some embodiments, an engineered bacterial cell may comprise a chromosome comprising (i) deletions of one or more genes selected from the group consisting of metL, tdh, dapA, and dhaM; and (ii) at least two copies of one or more genes selected from ppc, aspC, and pntAB. In some embodiments, one or more of the at least two copies of the one or more genes may be operably linked to a non-native promoter, such as, for example, a tac promoter. In some embodiments, an engineered bacterial cell may comprise a chromosome comprising (i) deletions of one or more genes selected from the group consisting of metL, tdh, dapA, and dhaM; and (ii) one or more of: (a) a ppc gene operably linked to a non-native promoter; (b) an aspC gene operably linked to a non-native promoter; and (c) a pntAB gene operably linked to a non-native promoter. In some embodiments, a non-native promoter may be a tac promoter.

In some embodiments, an engineered bacterial cell may comprise a chromosome comprising a deletion of one or more genes selected from the genes listed in Table 7. In some embodiments, an engineered bacterial cell may comprise a chromosome comprising an attenuated version one or more genes selected from the genes listed in Table 7. In some embodiments, an engineered bacterial cell may comprise a plasmid comprising a nucleotide sequence encoding one or more of the genes listed in Table 7. In some embodiments, and engineered bacterial cell may comprise a chromosome comprising at least two copies of one or more genes selected from the genes listed in Table 7.

### Methods of Engineering Threonine-Producing E. coli Strains

The present disclosure provides methods for “agnostic” bacterial strain engineering. Methods described herein may be applied to, for example, optimization of threonine production in bacterial cells, such as E. coli. In some embodiments, methods of bacterial strain engineering may comprise the following steps: (1) perform a pathway analysis; use general known gene pathway information to select genes whose inactivation or over-expression may increase production of a target molecule (e.g., threonine); (2) construct a set of bacterial strains comprising one or more over-expressed or inactivated genes; (3) collect threonine production data (and optionally RNA-Seq data) from the strains constructed in (2); (4) perform a computational analysis to generate new potential strains comprising combinations of two or more genetic modifications predicted to promote increased threonine production; (5) find additional new genes affecting production from metabolic modeling and ML analysis of expression data; and (6) construct and test a further set of engineered bacterial strains designs guided by artificial intelligence (AI).

In some embodiments, methods of bacterial strain engineering may comprise an algorithm. In some embodiments, an algorithm represents a sequence of prototyped processes. In some embodiments, a method of bacterial strain engineering may comprise a process comprising: (1) selecting genetic elements for strain engineering through Metabolic Modeling (MM), and identifying impactful genes as initial targets for strain engineering efforts; (2) designing engineered bacterial strains by selecting nucleotide sequences from one or more comparative genomics databases, choosing promoters to up- or down-regulate selected genes from baseline gene expression values, and designing primers and recombinant molecules for strain construction; (3) constructing a plurality of bacterial strains based on MM-guided designs obtained in (2) and their testing in Automated Lab by multiplex strain engineering, culture growth and production testing, and RNA-SEQ data collection: (4) performing a computational analysis using artificial intelligence (AI) data analysis comprising executing AI models from HTB data (Deep Learning for production data, Random Forest to RNA-SEQ), and MM filtering; (5) constructing and testing AI-designed improved strains based on the computational analysis of (4); and (6) iterating steps 4 and 5.

In some embodiments, the present disclosure teaches methods of predicting the effects of particular genetic alterations being incorporated into a given host strain. In some aspects, the disclosure provides methods for generating proposed genetic alterations that should be incorporated into a given host strain, in order for said host to possess a particular phenotypic trait or strain parameter. In some aspects, the present disclosure provides methods for using predictive models and/or computational algorithms to design novel bacterial strains with desired phenotype. In some embodiments, provided herein are methods for using predictive models and/or computational algorithms to design novel bacterial strains that are capable of producing threonine.

In some embodiments, the present disclosure provides methods for generating proposed genetic alterations that should be incorporated into a given host strain, in order for said host to possess an ability to overproduce threonine.

In some embodiments, the present disclosure teaches a system which generates proposed genetic modifications to host strains based on previous experimental data. In some embodiments, the recommendations of the present system are based on the results from the immediately preceding screening. In other embodiments, the recommendations of the present system are based on the cumulative results of one or more (e.g., one, two, three, or more) of the preceding screenings.

In some embodiments, the recommendations of the present system are based on scientific insights. For example, in some embodiments, the recommendations are based on known properties of genes (from sources such as annotated gene databases and the relevant scientific literature), codon optimization, or other hypothesis driven sequence and host optimizations.

In some embodiments, the proposed genetic modifications to a host strain recommended by systems or predictive models described herein are carried out by the utilization of molecular tools known in the art.

Provided herein are methods of engineering a threonine-producing bacterial cell. In some embodiments, a method of engineering a threonine-producing bacterial cell comprises: (i) identifying a set of optimized parameters predicted to result in increased production of threonine by a bacterial cell; (ii) constructing a plurality of bacterial strains, each bacterial strain comprising one or more of the optimized parameters of the set of optimized parameters identified in (i); (ii) collecting RNA-seq and threonine production data from the strains constructed in (ii); (iv) performing a computational analysis of the data collected in (iii) in order to obtain a further optimized set of parameters that predict increased threonine production; (v) repeating steps (ii), (iii), and (iv); and (vi) constructing one or more final bacterial strains, each bacterial strain comprising one or more of the optimized parameters of the set of optimized parameters identified in (v), thereby engineering a threonine-producing bacterial cell.

In some embodiments, a method of engineering a threonine-producing bacterial cell comprises identifying a set of optimized parameters predicted to result in increased production of threonine by a bacterial cell. In some embodiments, parameters may include intrinsic parameters (e.g., genome alterations) and/or extrinsic parameters (e.g., growth conditions). Non-limiting examples of parameters include background host strain, inactivated genes, overexpressed genes, presence of endogenous thrABC, chromosomal or plasmid localization of the modified threonine operon, induction of the threonine operon by Isopropyl β-d-1-thiogalactopyranoside (IPTG), growth time post-induction, total growth time, and culture medium. In some embodiments, optimized parameters may be selected based on existing data. For example, selecting genes for overexpression or inactivation for use as a parameter in a method of engineering a threonine-producing bacterial cell may be accomplished published or otherwise publicly available datasets.

In some embodiments, a method of engineering a threonine-producing bacterial cell comprises constructing a plurality of bacterial strains, each bacterial strain comprising one or more of the optimized parameters of the set of optimized parameters. In some embodiments, optimized parameters may have been identified in a prior step of such a method. Bacterial strains may be constructed using tools and techniques known in the art, and/or as discussed above.

In some embodiments, a method of engineering a threonine-producing bacterial cell comprises phenotypically characterizing initial constructed strains. In some embodiments. characterizing initial constructed strains may involve one or more of collecting threonine production data and collecting RNA-seq data from constructed strains. For example, the ability of a constructed strain to produce threonine may be quantified using tools and techniques known in the art, and as discussed in the Examples provided herein. RNA-seq data may be obtained from constructed strains using tools and techniques known in the art, and as discussed in the Examples provided herein. Experimental data obtained from constructed strains may be used to inform the selection of and/or optimization of parameters for one or more subsequent rounds of strain construction. Experimental data obtained from constructed strains may be used as input parameters for subsequent data analyses (such as, for example, computation analyses), which may be used to inform the selection of and/or optimization of parameters for one or more subsequent rounds of strain construction.

Methods of engineering a threonine-producing bacterial cells or bacterial strains may comprise computational approaches. Computational approaches may include, without limitation, machine learning (ML) approaches, metabolic modeling (MM), and RNA-seq analyses. In some embodiments, computational analyses may be used to guide engineering of novel threonine-producing bacterial strains.

In some embodiments, multiple rounds of parameter optimization may be employed in order to generate high-threonine producing bacterial strains. For example, steps of strain construction followed by steps of data (e.g., threonine production data and/or RNA-Seq data) collection may be repeated one, two three, four, five, or six or more times. One of skill in the art will be aware of the advantages and benefits to multiple rounds of data generation.

In some embodiments, methods of engineering a threonine-producing bacterial cell are useful for the identification and production of bacterial strains capable of producing substantial quantities of threonine when grown in culture. In some embodiments, when grown in culture, strains produced by the methods provided herein include strains which are capable of producing at least about 2 g/L to about 10 or more g/L of threonine in about 24 hours. In particular, when grown in culture, strains produced by the methods provided herein include strains which are capable of producing at least about 2 g/L, at least about 3 g/L, at least about 4 g/L, at least about 5 g/L, at least about 6 g/L, at least about 7 g/L, at least about 8 g/L, at least about 9 g/L, at least about 10 or more g/L of threonine in about 24 hours. In some embodiments, strains produced by the methods provided herein are capable of producing at least 2.0 g/L, at least 3.0 g/L, at least 4.0 g/L, at least 5.0 g/L, at least 6.0 g/L, at least 7.0 g/L, at least 8.0 g/L, at least 9.0 g/L, or at least 10.0 or more g/L of threonine by about 24 hours of growth in culture. In some preferred embodiments, methods of engineering a threonine-producing bacterial cell are useful for the identification and production of bacterial strains capable of producing at least 8.0 g/L of threonine by about 24 hours of growth in culture.

### EXAMPLES

These examples are provided for illustrative purposes only and not to limit the scope of the claims provided herein.

### Example 1. General Methods

### Growth of Bacterial Cells in 96 Well Plates for Threonine Production

Axygen 1. 1 mL deep well plates were used to grow the cultures using a QuickSeal breathable membrane from Thomas Scientific. The cells were incubated at 37° C., with approximately 80% relative humidity at 1000 rpm on a Labforce microplate shaker (Thomas Scientific). The strains for threonine production measurements were frozen in 96 well plates. The plates were laid out in such a way that the plates contained a duplicate set of the cell cultures. This was done so that one half of the plate would contain un-induced cells and the other half of the plate would contain cells that had were induced with IPTG for expression of threonine. Cells were started directly from the frozen cultures by transferring 5 mL of the frozen culture to 200 ml of LB (10 g/L1 bactotryptone, 5 g/L, yeast extract, 10 g/L NaCl) with the appropriate antibiotics and grown overnight at 37° C. On the next day, 20 ml of LB μculture was transferred to 200 μL of minimal seed media (KH2PO4 1 g/L, Bis-Tris 40 g/L, (NH4)2SO4 10 g/L, Glucose, 7.5 g/L, MgSO4·7H2O 0.3 g/L, adjusted to pH 7.0) containing proline (300 mg/ml), isoleucine (100 mg/ml), methionine (200 mg/ml), lysine (100 mg/ml), diaminopimelate (100 mg/ml), and thiamine (1 mg/ml) with appropriate antibiotics. After about 24 hr, 20 μL was transferred to 200 μL of minimal fermentation media (KH2PO4, 1 g/L, Bis-Tris, 40 g/L, (NH4)2SO4, 30 g/L, Glucose, 30 g/L, MgSO4·7H2O, 1.2 g/L, Na3Citrate, 1.0 g/L, MnSO4·H2O, 0.02 g/L, FeSO4, 0.03 g/L. Adjusted to pH 7.0) containing the same amino acids and thiamine as the seed media but without any antibiotics. At 5 hr, IPTG, at a final concentration of 1 mM, was added to one half of the duplicated plate to induce the threonine operon. When several time points were required multiple 96 well plates were inoculated at the fermentation stage and the entire plate was removed for samples. When the plate was sampled, growth was measured by reading the optical density of 10-fold dilutions in a Hidex plate reader at 600 nm. The cells were then spun down and about 150 ml of supernatant was removed for measurement of threonine and stored at −20 C.

### Growth of Cells in Flasks for RNAseq Measurements

Cells were struck out on LB agar plates with appropriate antibiotics. The cells from the plates were then inoculated directly into 250 ml baffled flasks containing 20 mL minimal seed media with proline, isoleucine, methionine, lysine and thiamine. For simplicity and consistency, all strains received the same amino acid additions whether they required them or not. Appropriate antibiotics were added. After approximately 20 hr, 2 mL was transferred to two duplicate flasks containing 22 mL of the fermentation media containing amino acid additions and no antibiotics. At 5 hr, IPTG, at a final concentration of 1 mM, was added to induce the threonine biosynthetic enzymes. At indicated time points, the growth was measured at 600 nm and 1 mL of cells was spun down for 15 seconds in a microcentrifuge, and the supernatant poured off. The supernatant and the cells were frozen at −80 ° C. The cells were used to prepare RNA and the supernatant for threonine measurements.

### Threonine Measurements

Threonine was measured using the BioVision PicoProbe Threonine Assay Kit (Fluorometric). The protocol was modified for use in 384 well plates. The assay was scaled down to 20 mL instead of 100 mL. The threonine samples from flasks or 96 well plates were diluted 100-fold to be in the linear range of the assay. Standard curves were run on each 384 well plate.

### Glucose Measurements

Glucose was measured using the Sigma-Aldrich glucose assay kit (GAGO20) scaled down for use in 96 well assay plates (Corning Clear Flat Bottom Assay Plate #9017). Each well contained 70 μl assay mix 5 μl sample (10-2 dilution). Each plate had a glucose standard curve for calculating glucose concentration.

### RNA Extraction and Sequencing

RNA was extracted using the Qiagen RNeasy PowerMicrobiome Kit and quality-checked with the Agilent 2100 Bioanalyzer before library preparation. Initially, each sample had an RNA integrity number (RIN) assigned using the Bioanalyzer. After assessing the quality, all samples were quantified to obtain RNA concentrations using a microplate reader (Tecan) and the Quant-iT RNA Assay Kit (Invitrogen). Ribosomal RNA was then removed using the bacterial FastSelect 5S/16S/23S kit (Qiagen). After removal of rRNA, cDNA sequencing libraries were made using the KAPA RNA HyperPrep Kit (Roche). Sequencing libraries were then assessed using the Bioanalyzer. All samples were quantified using the Qubit dsDNA HS Assay kit (Invitrogen). Samples were then pooled in an equimolar fashion for sequencing. Once libraries were pooled, they were denatured according to Illumina's recommendations, and loaded on the sequencer. Sequencing was completed on either the Illumina MiSeq on a 2×251 bp run or Illumina HiSeq2500 on a 2×151 bp run to achieve around 2 million reads per sample.

The RNA Seq expression data was processed using the FASTQ Utilities service of PATRIC (Davis, J. J., et al., The PATRIC Bioinformatics Resource Center; expanding data and analysis capabilities. Nucleic Acids Res, 2020. 48(D1); p. D606-D612) for read trimming and quality control, and the PATRIC RNA Seq service to assemble the reads and align them to the MG1655 reference genome using the Tuxedo strategy (the Cufflinks workflow) (Trapnell, C., et al., Differential gene and transcript expression analysis of RNA-seq experiments with TopHat and Cufflinks. Nat Protoc, 2012. 7(3); p. 562-78). The FPKM scores were converted to TPMs to produce gene expression scores which could be compared across different samples.

A set of tools supporting storage, visualization, and analysis of data generated in the project were developed. They available at maseq.theseed.org. These tools allow to analyze expression data, and to generate and compare differential-expression profiles, display time-courses and convert numeric expression and differential expression values into categories. It links RNA data and analytical tools to information on gene functions including their placement in functional subsystems (Overbeek, R., et al., The SEED and the Rapid Annotation of microbial genomes using Subsystems Technology (RAST). Nucleic Acids Res, 2014. 42 (Database issue); p. D206-14.), operon and regulon information (including i-modulons developed by (Sastry, A. V., et al., The Escherichia coli transcriptome mostly consists of independently regulated modules. Nat Commun, 2019. 10(1); p. 5536). It also represents an open environment which was designed to support similar studies.

### Plasmid Construction

All plasmids were generated via Gibson assembly cloning using the NEB Gibson Assembly cloning kit or the NEBuilder Hifi DNA Assembly kit. Q5 DNA polymerase from NEB was used for all PCR reactions. All PCR reaction products were treated with DpnI to cut any plasmid or chromosomal DNA. Finally, PCR fragments were cleaned up using the NEB Monarch PCR clean up kit. DNA concentrations were determined using a NanoDrop™ spectrophotometer. All plasmids were sequenced.

Three core plasmids were constructed containing the threonine pathway genes, thrABC, and asd genes. The thrABC and the asd genes were cloned into modified vector pSR58.6 (Schmidt, Sheth et al. 2014), which resulted in a plasmid containing colE1 origin, the chloramphenicol resistance gene, with the thrABC-asd-gfp operon controlled by Tac promoter, and lacIq gene. The resulting three constructs comprise the following:
Plasmid pwt2.1.1 comprised the wild type sequence of the thrABC operon and the a sequence of asd containing one amino acid change (G8S) relative to wild type asd.
Plasmids pfb6.4.2 and pfb6.4.3 comprised a feedback-resistant thrA gene, with a G433R mutation (from ATCC21277). Plasmid pfb6.4.3 contained two copies of the lacIq gene, which increased the growth rate of plasmid-carrying strains when compared with pfb6.4.2, which contains only one copy of the lacIq gene. Strains carrying pfb6.4.3 grew significantly faster than the pfb6.4.2 strains.

Supplemental plasmids were constructed by cloning the following genes: E. coli genes, rhtA, zwf, aspC, ppc, aceBA, pntAB and a E. coli codon-optimized, Rizobium etli pyc gene (Gokarn 1999) were cloned in a modified vector pSR43.6 (Schmidl, Sheth et al. 2014). Resulting plasmids contained the p15A origin, spectinomycin resistance gene and the gene of interest controlled by a constitutive promoter J23108 (Moore, Lai et al. 2016).

### Chromosomal Deletions

Chromosomal deletions were made in strains MG1655 and ATCC 21277 using the Gene Bridges Quick & Easy E. coli Gene Deletion Kit and following their protocol. The deletions were made to remove the entire coding region from the start codon to the stop codon and selected for by resistance to kanamycin. They were checked by PCR to determine that the kan gene was inserted in the correct spot. The deletions were moved to different strains and combined with other deletions by P1 transduction. Kan genes were flipped out using the flanking fit sites according to the Gene Bridges protocol.

### Genome Insertions

The Tac promoter was inserted into the genome of MG1655 and ATCC21277, 5′ of the thrABC genes using the Gene Bridges Quick & Easy E. coli Gene Deletion Kit. For the thrABC genes, a construct was designed so that the Tac promoter and ribosome binding site (RBS) are controlling the expression of the thrABC genes. The Tac promoter and the RBS were the same as the ones used in the core plasmids (pfb6.4.2, pfb6.4.3). The threonine leader peptide (thrL) and sequences 5′ of thrL were deleted from the chromosome when the tac promoter and RBS were inserted. Finally, the kan marker gene was flipped out. The final constructs were verified by DNA sequencing. The same tac promoter and RBS were inserted into the chromosome to control the asd gene. Additionally, the region 5′ of the asd gene was deleted. Two versions of the tac promoter controlling the thrABC genes (ptacthrABC) were produced: one in MG1655 where the thrA gene was wild type and one in ATCC21277 where the thrA gene was feedback-resistant to threonine (thrA*). The wild type ptacthrABC was moved from MG1655 into ATCC21277 by P1 transduction, to make a ATCC21277 with a wild type ptacthrABC. The sequence was verified by DNA sequencing. Conversely, the ptacthrA*BC from ATCC21277 was moved into MG1655 also by P transduction to make a MG1655 with a feedback resistant thrA gene controlled by the tac promoter. P1 transduction was also used to move ptacthrABC into several other MG1655 strains with various deletions.

### P1 Transduction

P1 transduction was done with P1vir using protocols described in (Miller, J. H. (1992). A short course in bacterial genetics: a laboratory manual and handbook for Escherichia coli and related bacteria, New York (N.Y.); Cold Spring Harbor laboratory press, Vol. 1 p 263-275.), which is incorporated by reference herein in its entirety.

### Metabolic Modeling

The transcriptome data from chromosomal mutants were incorporated into the iML1515 E. coli metabolic model in order to define condition specific results. FPKM data were used to remove lowly expressed genes in the base strain (no added constructs). The experimental data from the base strain were used to set the biomass and threonine synthase constraints to obtain a reference solution and solved using parsimonious FBA to reduce overall flux through the system. The reference solution was then multiplied by the fold change obtained from the RNASeq data of a particular construct vs. the base strain to obtain a target solution. A modified version of linear MOMA (Minimization of Metabolic Adjustments) was performed to obtain a metabolically feasible solution reflecting the target fold changes (fit target solution). Approach 1 modeled overexpression and knockout/knockdown by adjusting the upper or lower bounds of the reaction by a factor based on the copy number of genes per reaction (e.g. if two genes encode the same reaction, the upper bounds of the reaction would be reduced to half of the fit target solution). Fluxes of threonine and biomass predicted by this approach were compared to experimental values. When looking at true/false positives/negatives, an increase in threonine production was defined as a 5% increase. To analyze all genes systematically, approach 1 was used. The genes that could increase threonine production by at least 5% while maintaining biomass growth and had some flux in the base strain (>1e-5) were reported. In approach 2. the threonine and biomass fluxes in the model were constrained to experimental values and the fluxes of those reactions associated with genes which were engineered were compared to their expected values (e.g. a knockout should result in reduced or no flux). When examining the top producing mutants, the biomass was constrained to experimental values and the THRD reaction (tdh) was knocked out.

### Machine Learning Models

A deep neural network was used to predict threonine production from combinations of strain engineering elements shown in Table 3. The feature vector consisted of indicators for individual strain modifications, with multi-valued modifications like the core threonine operon specification, one-hot encoded for a total of 33 input dimensions and one output dimension (threonine yield). When multiple experiments were performed on identical samples, the trimean of all results was used as the target.

DeepLearning4J was used for model training, prediction, and hyperparameter tuning. For most models, a batch normalization layer followed by 2 to 0 feed-forward layers with no gradient normalization and an output layer loss function of L2 (squared error) were used. The hyperparameter searches were optimized for lowest mean absolute error. The activation functions tested were hard hyperbolic tangent, rectified linear unit. Gaussian error linear unit, and the normalized exponential function Soft max. The same function was used for all inner layers, but a second function was sometimes used for the input layer. The model was validated using 10-fold cross-validation. The mean error during cross-validation was 3.1% of the total output range, with an IQR equal to 1.0% of the total output range.

### General Algorithm for AI-and-MM Driven Organism Engineering for Industrial Bioproduction

The study described in the following Examples demonstrates that engineering of industrial strains based on general computational tools and “general” metabolic and comparative genomic information can produce high-yielding microbial strains in predictable and accelerated manner. Strains were constructed which produced up to 2.5 times more threonine than a strain only recently used in actual industrial production (not in the conditions of industrial fermentation, but with enough glucose, buffered media, and sufficient aeration). The study represents a prototype, which can be transferred to an AI-driven autonomous lab capable to perform organism engineering in a highly automated manner by performing a general algorithm. Such algorithm represents a sequence of prototyped processes:
1) Selection of genetic elements for strain engineering by Metabolic Modeling (MM), which utilize metabolic models to systematically simulate the knockdown and induction of every metabolic gene, and identify most impactful genes as the initial targets for strain engineering efforts;
2) Computerized strain engineering planning by selecting nucleotide sequences from comparative genomics database, choosing promoters to up- or down-regulate selected genes from baseline gene expression values, and providing exact design of primers and recombinant molecules:
3) Experimental execution of MM designs and their testing in Automated Lab by multiplex strain engineering, culture growth and production testing, and RNA-SEQ data collection;
4) AI data analysis and design of optimal combination of elements by building AI models from HTB data (Deep Learning for production data, Random Forest to RNA-SEQ), and MM filtering. A key element here is the application of AI models to a virtual space of possible engineering combinations to predict a next set of improved strains;
5) Construction and testing AI-designed improved strains; and
6) Iterating step 4 and 5.

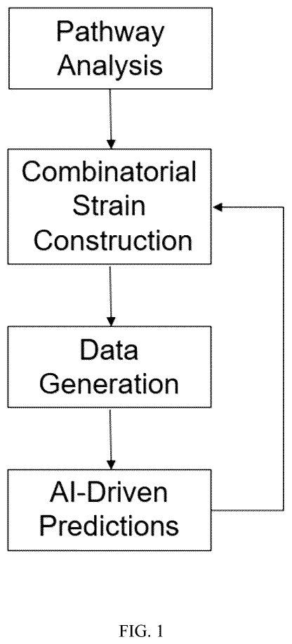

Accelerated “agnostic” strategies for bacterial strain engineering presented herein are based on systems biology, metabolic modeling, and machine learning. Algorithms provided herein may be used or performed according to the scheme shown in FIG. 1.

### Example 2. Selection of Bacterial Hosts

Type strain of E. coli K12, MG1655, was re-sequenced together with 8 known threonine producing strains: ATCC21278 (Shiio 1971), ATCC21277((Shiio 1971)), NRRL B-21593 (Wang 1999), NRRL B-30319 (Liaw 2007), NRRL B-30823(8) (D'Elia 2010), NRRL B-30316 (Liaw 2007), NRRL B-30317 (Liaw 2007), NRRL B-30318 (Liaw 2007), which belong to three different lineages. Genomes were assembled into 100-150 contigs separated by rRNAs and other repeated elements and annotated using PATRIC pipeline. The sequencing error rate was below 10%, when compared with Gene Bank version of MG1655. Strains carry from 110-370 non-silent mutations. Only 14 mutations were present for all strains. Upstream regions carried 23 mutations, which were present in all sequenced strains.

Two strains, MG1655 (a wild type), and ATCC21277 (which carry mutations increasing threonine production to an intermediate level) were chosen as hosts for all subsequent experiments.

### Example 3. Initial Selection of Genes for E. coli Strain Construction

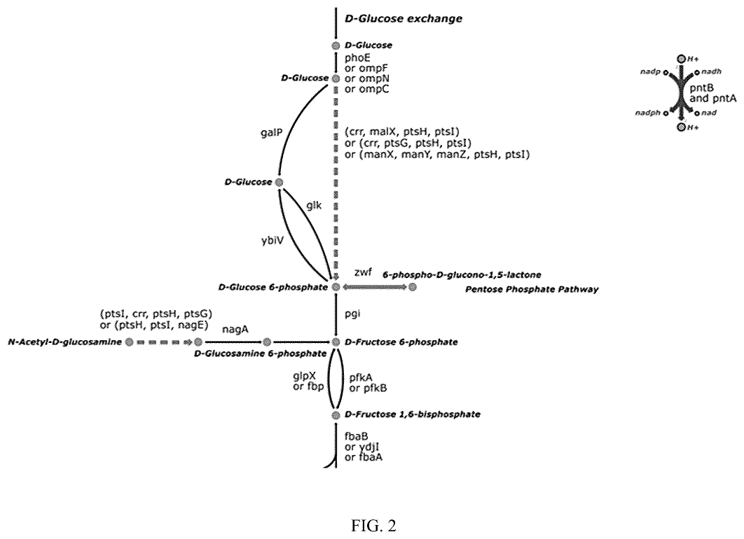

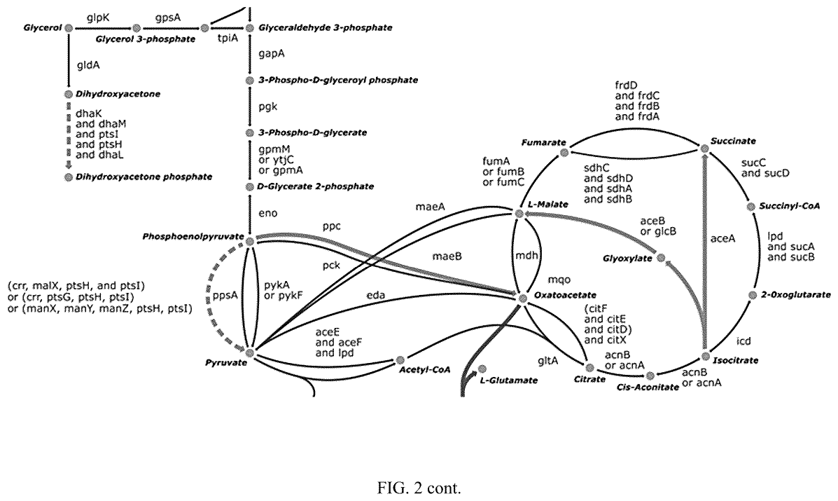

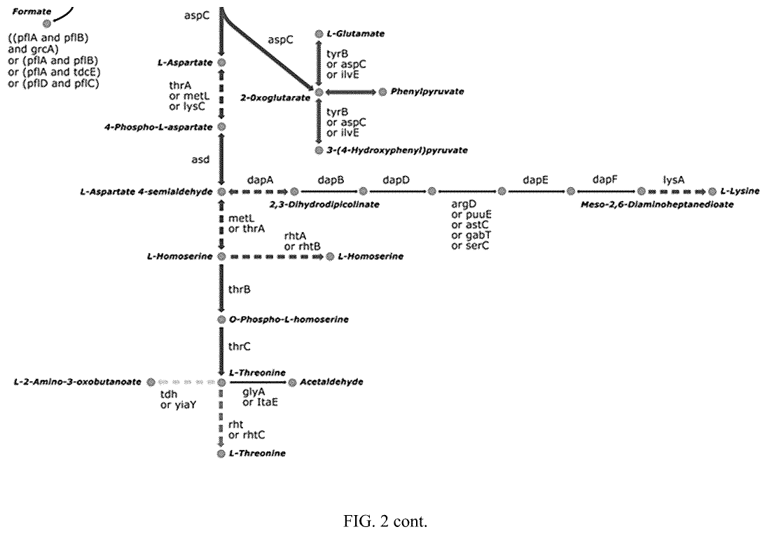

Sixteen genes selected based on their potential impact on threonine biosynthesis are shown in Table 1, Genes were selected using publicly available data in the Pathosystems Resource Integration Center (PATRIC). These genes belong to five functional blocks (1) carbon backbone; (2) threonine biosynthesis; (3) competing pathways; (4) threonine catabolism (degradation), and (5) threonine transport. These genes, inter alia, are shown in FIG. 2.

TABLE 1
Genes used for engineering of initial strains

| Gene | Bigg ID | Enzyme Name | Type | Expected Effect | Reference |
|---|---|---|---|---|---|
| thrABC | ASPK, HSDy, | Aspartokinase/ | Threonine | Amplification | (Kozlov Iu, |
| | HSK, THRS | I-homoserine | biosynthesis | leads to | Kochetova |
| | | dehydrogenase I, | | overproduction of | et al. 1980) |
| | | homoserine | | theonine | |
| | | and threonine | | | |
| | | synthetase | | | |
| asd | ASAD | Asparatate | Threonine | Amplification | (Debabov |
| | | semialdehyde | biosynthesis | leads to | 2003) |
| | | dehydrogenase | | overproduction of | |
| | | | | theonine | |
| pyc | PC (not in | pyruvate | Carbon | Increase the flow | (Peters- |
| | map) | carboxylase | backbone | of carbon to | Wendisch, |
| | | | | oxaloacetate and | Schiel et al. |
| | | | | asparatate, a major | 2001) |
| | | | | bottleneck for | |
| | | | | threonine | |
| | | | | production in C. | |
| | | | | glutamicum | |
| rhtA | HOMt2pp, | Threonine exporter | Threonine | Overexpression | (Livshits, |
| | THRt2pp | | export | improves | Zakataeva |
| | | | | threonine | et al. 2003) |
| | | | | production | |
| zwf | G6PDH2r | glucose-6-phosphate | Carbon | Increasing | (Becker, |
| | | dehydrogenase | backbone | NADPH. | Klopprogge |
| | | | | Boisynthesis of | et al. 2007) |
| | | | | threonine requires | |
| | | | | 3 NADPH | |
| | | | | molecules/ | |
| | | | | threonine | |
| | | | | molecule. | |
| pntAB | THD2pp | transhydrogenase | Other | Coverts NADH to | (Liu, Li et |
| | | | | NADPH. | al. 2019) |
| | | | | Boisynthesis of | |
| | | | | threonine requires | |
| | | | | 3 NADPH | |
| | | | | molecules | |
| | | | | threonine | |
| | | | | molecule. | |
| lysC | ASPK | Aspartokinse- | Threoine | lysine feedback | (Ogawa- |
| | | homoserine | biosynthesis | resistant allele | Miyata, |
| | | dehydrogenase | | improves | Kojima et |
| | | | | threonine | al. 2001) |
| | | | | production. | |
| aceBA | ICL, MALS | Isocitrate lyase and | Carbon | Increased | (Liu, Li et |
| | | malate synthase | backbone | expression of the | al. 2019) |
| | | | | glyoxylate shunt | |
| | | | | improves | |
| | | | | threonine | |
| | | | | porduction | |
| aspC | ASPTA, PHETA1, | Threonine | Threonine | Over-expression | (Zhao, Lu |
| | TYRTA, CYSTA | aminotranferase | biosynthesis/ | improves | et al. 2020) |
| | | | Competing | threonine | |
| | | | pathways | production | |
| metL | ASPK, HSDy | Aspartokinse | Threonine | Homologue to | (Neidhardt |
| | | homoserine | biosynthesis | thrA gene. Not | and Curtiss |
| | | dehydrogenase | | Highly expressed | 1996) |
| | | | | in E. coli | |
| lysA | DAPDC | Diaminopimelate | Competing | Making more | (Lee, Park |
| | | decarboxylase | pathways | precursors | et al. 2007) |
| | | | | available for | |
| | | | | threonine | |
| | | | | biosynthesis | |
| tdh | THRD | Threonine | Threonine | Knocking out | (Lee, Park |
| | | Dehydrogenase | catabolism | threonine | et al. 2007) |
| | | | | degradation | |
| | | | | pathways | |
| ptsG | ACGAptspp, | Phosphotranseferase | Carbon | Deletion of the | (Zhu, Fang |
| | GLCptspp | | backbone | PTS system for | et al. 2019) |
| | | | | glucose uptake | |
| | | | | expected to | |
| | | | | improve | |
| | | | | threonine | |
| | | | | production | |
| dhaM | DHAPT | Dihydroxyacetone | Carbon | Knock out of | |
| | | kinase | backbone | dhaM improves | |
| | | phosphotransferase | | growth of PTS | |
| | | component | | deleted strains | |
| dap4 | DHDPS | Dihydrodipicolinate | Competing | Deletion of dapA | |
| | | synthase | pathways | gene may improve | |
| | | | | precursor flow to | |
| | | | | threonine. | |
| ppc | PPC | Phosphoenolpyruvate | Carbon | Over-expression | (Lee, Park |
| | | carboxylase | backbone | of ppc improves | et al. 2007) |
| | | | | threonine | |
| | | | | production | |

Gene Name is the standard name of the gene. Note that the full theronine operon (thrA, thrB, thrC) is listed once. These three genes were treated as a single unit when creating mutations.
Bigg ID lists the Bigg identifiers for the associated reactions.
Enzyme Name is the annotation used on the PATRIC web site.
Type describes the metabolic subsystem containing the gene.
Expected Effect is a short description of how over-expressing or knocking out the gene is expected to affect threonine production.
Reference describes the paper in which the gene's function is described.

### Example 4. Characterization of Initial Strains

E. coli genes, rhtA, zwf, aspC, ppc, aceBA, pntAB and codon-optimized Rhizobium etli pyc gene (Gokarn 1999) were cloned in a modified vector pSR43.6 (Schmidl, Sheth et al. 2014). The resulting plasmids contained the p15A origin, spectinomycin resistance gene and a gene of interest controlled by a constitutive promoter J23108 (Moore, Lai et al. 2016).

The thrABC and the asd genes were cloned into modified vector pSR58.6 (Schmidl, 2014), which resulted in a plasmid containing colE1 origin, the chloramphenicol resistance gene, and a synthetic operon thrABC-asd-gfp controlled by tac promoter, and lacIq gene. Three variants were engineered:
1) pwt2. 1.1, which comprises the wild type sequence of the thrABC operon and the sequence of asd harboring one amino acid change (G8S) relative to the wild-type sequence;
2) pfb6.4.2, which contains a feedback resistant thrA gene (with a G433R mutation) from ATCC21277; and
3) pfb6.4.3, which comprises two copies of the lacIq gene, which increases the growth rate of plasmid-carrying strains when compared with pfb6.4.2.

Eight individual genes, eleven combinations of two genes, and three combinations of three genes (all genes used are shown in Table 1) were deleted in MG1655 using 1red gene-replacement system described in (Datsenko, 2000) Genes replaced by Km resistance marker in MG1655 were moved to ATCC21277 using P1 transduction (Thomason, Costantino et al. 2007). The table at maseq.theseed.org/html/big_production.html contains all of the samples and strains tested in the study, including the predicted production (where applicable), the actual production, the optical density measurement, and the glucose utilization where known.

Constructed strains represent combinations of (1) individual or combined deletions of one to three out of 8 E. coli genes, (2) two host strains, (3) three modifications of Thr operon (inserted in chromosome or cloned in plasmid), and (4) 7 cloned and overexpressed “supplementary' genes. These modifications, and other factors varied in collected samples are listed in Table 2 and are reflected in samples names as shown in the Table 2.

TABLE 2
Strain naming key.

| | | | Copy | | | Deleted | | ime | |
|---|---|---|---|---|---|---|---|---|---|
| | | | number | | | genes | | point, | |
| | | | for | | Added | (with″D″ | | hours | |
| | Status | | modified | | (overex- | added | Thr | or | |
| | original | Re-designed thr | thr | Asd | pressed) | to gene | operon | growth | |
| Host | thrABC | operon | operon | status | genes | name) | induction | stage | Media |
| 7- | D - | 0 - Non-modified | P - | asdD - | aceBA, | dapA, | 1 - | N | M1 - |
| ATCC212 | deleted | T - chromosomal | Plasmid | deleted | aspC, | dhaM, | IPTG+ | ML- | synthetic |
| 77 | 0 - not | copy of the thrABC | C - | asdO - | pntAB, | lysA, | 0 - | midLog | fermentation |
| M- | | operon under under | Chromos- | original | ppc, | lysC, | IPTG - | | media |
| MG1655 | | control of the tac | ome | asdT- | pyc, | metL, | | | M2- M9+ |
| used for | | promoter (without | 0 - Non- | chromoso- | rhtA, | ptsG, | | | pro- thi- |
| engineering | | asd) | modified | mal copy | zwf | rhtA, | | | ile- met |
| Full | | TAI - chromosomal | A - thrA | of the asd | | tdh | | | thr |
| ATCC | | copy of the thrABC | allosteric | gene | | | | | M3- M9+ |
| names for | | controlled by the tac | regulation | under | | | | | pro- thi |
| the rest of | | promoter with a | removed | control of | | | | | ile- met |
| strains | | feedback resistant | | the tac | | | | | |
| | | thrA gene | | promoter. | | | | | |
| | | Tasd - operon with | | | | | | | |
| | | tac promoter and asd | | | | | | | |
| | | added | | | | | | | |
| | | TasdA - same | | | | | | | |
| | | above, plus thrA | | | | | | | |
| | | allosteric regulation | | | | | | | |
| | | removed | | | | | | | |
| | | TasdA1 - the same as | | | | | | | |
| | | above with thrA | | | | | | | |
| | | allosteric regulation | | | | | | | |
| | | removed and stronger | | | | | | | |
| | | regulated tac by two | | | | | | | |
| | | copies of lacIq | | | | | | | |

Individual effects of many of these genes on threonine production has been already studied, as shown in the references in the Table 1. However, effects of combinations of such modifications can be derived from the effects of individual modifications and must be manually tested.

In total, 649 strains carrying such combinations were constructed. Their composition (presence or absence of modified or deleted genes) was verified using PCR. 2984 samples, representing different time points with and without IPTG were grown in 96-deep-well plates in synthetic media. Their growth yield and threonine production were measured. Samples producing above 2 g/L of threonine are shown in Table 3.

TABLE 3
Initial modified strains producing more than 2 g/L of threonine.

| | Threonine, | |
|---|---|---|
| Modification Profile | g/L | OD600 |
| 7_0_TA1_C_asdT_pyc_DmetLDtdh_I_24_M1 | 2.9050 | 6.6100 |
| 7_0_TA1_C_asdT_pntAB_DmetLDtdh_I_24_M1 | 2.8113 | 5.6525 |
| 7_0_TA1_C_asdO_pntAB_DmetLDtdh_I_24_M1 | 2.6910 | 6.6967 |
| 7_0_TA1_C_asdT_aspC_DmetLDtdh_I_24_M1 | 2.5650 | 4.9762 |
| 7_0_TA1_C_asdO_pntAB_DlysCDtdh_I_24_M1 | 2.4446 | 7.6133 |
| 7_0_TA1_C_asdO_pyc_DlysCDtdh_I_24_M1 | 2.3695 | 6.9300 |
| 7_0_TA1_C_asdT_000_DmetLDtdh_I_24_M1 | 2.3312 | 5.8500 |
| 7_0_TA1_C_asdO_pyc_Dtdh_I_24_M1 | 2.2601 | 7.4500 |
| 7_D_TasdA1_P_asdD_pyc_DmetLDtdh_I_24_M1 | 2.2515 | 5.1500 |
| 7_0_TA1_C_asdO_ppc_Dtdh_I_24_M1 | 2.2491 | 5.1009 |
| 7_0_TA1_C_asdO_pntAB_Dtdh_I_24_M1 | 2.1895 | 4.7200 |
| 7_0_TA1_C_asdO_aspC_DmetLDtdh_I24_M1 | 2.1830 | 5.7600 |
| 7_0_TA1_C_asdO_pyc_DmetlLDtdh_I_24_M1 | 2.1427 | 7.2900 |
| 7_0_TA1_C_asdO_aspC_Dtdh_I_24_M1 | 2.1238 | 5.2546 |
| 7_0_TA1_C_asdT_ppc_DmetLDtdh_I_24_M1 | 2.1209 | 3.8000 |

### Example 5. Observed Effects of Individual Modifications

### Gene Knockouts

MetL deletion lead to an increase in threonine production. MetL, like thrA, codes for both aspartokinase and homoserine dehydrogenase. Accordingly, it was surprising that a knockout of these enzymatic activities promoted the synthesis of threonine.

DhaM deletion resulted in improvement in threonine production in a majority of the generated strains. dhaM codes for dihydroxyacetone kinase and uses PEP for phosphorylation DHA. This protein is part of the PTS system but does not transport DHA. Rather, it only phosphorylates DHA. In knockouts of ptsI, which grow very slowly, it was observed that faster growing colonies bad additional mutations in dhaM and dhaR.

Tdh codes for threonine dehydrogenase and is one of the threonine degradation pathways. It is known that a knockout of tdh improves threonine production. In constructed strains, deletion of tdh produced mixed results.

LysC, dapA, rhtA did not improve threonine production in most cases.

### Overexpressed Genes

PntAB codes for a membrane bound transhydrogenase that interconverts NADH to NADPH. Three NADPH molecules are required for the biosynthesis of each threonine molecule. Overexpression of pntAB robustly increased threonine production, indicating that lack of reduction may be a critical limitation in threonine production.

Ppc codes for phosphoenolpyruvate (PEP) carboxylase, a key enzyme in threonine production, which converts PEP to oxaloacetate (OAA). Overexpression of ppc increased threonine production.

AspC codes for aspartate aminotransferase. It converts OAA to aspartic acid using glutamine as the amino donor. Overexpression of aspC increased threonine production.

RhtA is a known exporter of threonine, and its overexpression is believed to leads to increased production of threonine. Overexpression of rhtA led to a decrease in threonine production.

Zwf codes for glucose dehydrogenase which produces NADPH. It was expected that overexpression of zwf may increase threonine production. However, in the present study, overexpression of zwf caused a decrease in threonine production, likely due to too much carbon flowing down the pentose phosphate pathway.

AceBA codes for isocitrate lyase and malate synthase. These are the enzymes in the glyoxylate shunt. Overexpression of this pathway was implementated to recapture carbon that was converted to acetyl-CoA for threonine biosynthesis. Overexpression of these two enzymes did not increase threonine production.

Pyc codes for pyruvate carboxylase, an enzyme not normally expressed in E. coli. The Pyc enzyme converts pyruvate to oxaloacetate (OAA) a key intermediate in threonine production. In E. coli, pyruvate is essentially lost for threonine production, so by adding pyruvate carboxylase, one would expect to recover some of this carbon for threonine production. However, overexpression of Pyc did not promote increased threonine production in tested strains.

In summary, ATCC21277, in which upregulated feedback resistant threonine operon and one of the several “positive” modifications was integrated, was generated that was able to produce up to 2.7 g/L of threonine when grown in minimal media in microtiter plates. This production level approaches rates of industrial threonine-producing strains which generate up to 3.5 g/L in the same conditions. Some modifications contributing to high threonine production include:
1) chromosomal location of IPTG-induced threonine operon (up to 2.6 g/L), whereas the best-producing variant with thrABC-asd on a plasmid produce 1.3 g/L).
2) addition of overexpressed ppc, pntAB or aspC; and
3) deletions of tdh, and metL and lysC. Somewhat surprising are the observations that constructs with upregulated asd, zwf and rhtA genes are not seen in top threonine production variants.

### Example 6. Metabolic Modeling to Simulate Individual Modifications and Modification Combinations

Metabolic modeling approaches were applied to test how well metabolic models agreed with experimental observations for the impact of individual and combined deletions and overexpressions observed in the initial constructed strains. A MOMA approach was used for this analysis. Briefly, a wild-type strain was modeled, which was then perturbed to simulate strain modifications (e.g. reducing or eliminating flux through reactions associated with gene knockouts and increasing flux through reactions associated with overexpressed genes). A new flux solution that minimizes the perturbation from the wild-type flux was then computed.

Of the knockouts attempted, the models correctly predicted that one of these would increase threonine production, as they eliminate lysA, a pathway that competes with threonine biosynthesis. The models mostly agreed with experimental results on over-expression of certain genes when they had no impact on threonine production, but failed to predict the beneficial effect that two over-expressed genes had on threonine production. The one case in which models did predict a beneficial effect of overexpression, with aceBA, turned out to be incorrect.

To explore other possible useful knockouts or overexpressions, the above approached were systematically to predict the impact of individual knockout or overexpression of all 1515 genes included in the iML1515 E. coli model. Here, using the M_0_TA1_C_asdT background, an additional 36 potential knockdown/outs and 91 potential over-expressions that could potentially enhance threonine production (>5% increase) while maintaining growth were predicted. These results were considered for potential targets for future study. Using the 7_0_TA1_C_asdT background, only 16 reactions were predicted, seven of which were in the threonine biosynthesis pathway.

The above approach was applied to simulate all initial combinations of modifications attempted in Examples 3 and 4 above. However, in this instance, the accuracy of the models declined substantially to 47%, with more false negatives than false positives. This indicated that this particular modeling approach was not very effective at predicting the impact of multiple simultaneous modifications, most likely because MOMA as a method is designed to handle small perturbations rather than the large ones, as performed here. In addition, very little quantitative agreement between threonine production and growth rate predictions was found from the MOMA method, compared with experimentally measured values. Correlations in quantitative threonine and biomass predictions were 0.07 and 0.008 respectively. In the case of MetL, models predicted that a knockout would reduce threonine, thus the models provide no indication that this knockout may be at all beneficial for threonine production.

### Example 7: Validation of Artificial Intelligence (AI) Ability to Predict Threonine Production

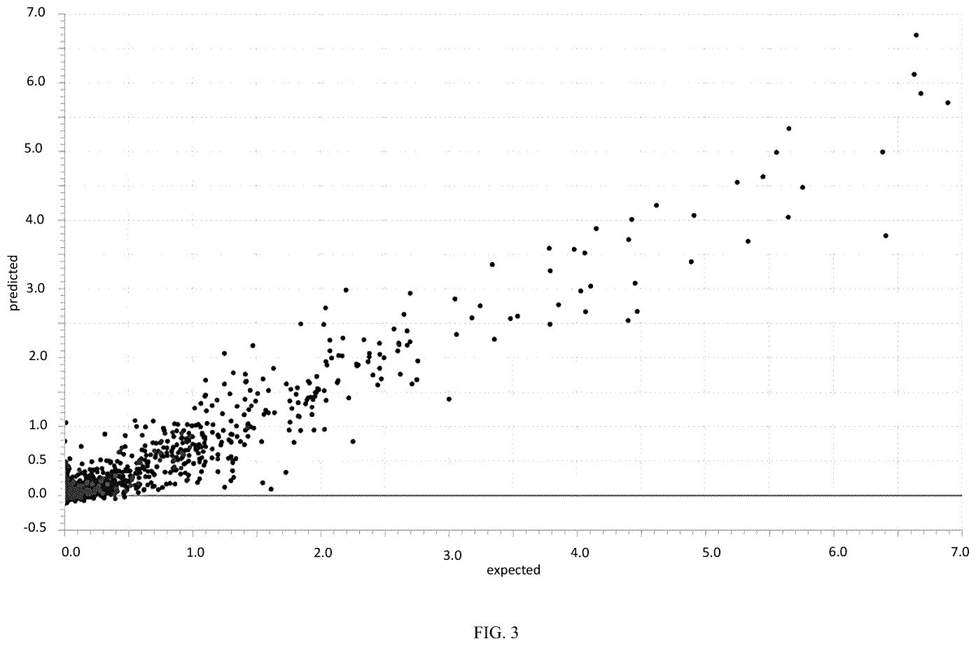

A training set of 2749 samples, which was described in the previous section, was analyzed using a deep learning neural network to predict threonine production from combinations of strain-engineering elements used as descriptive attributes (features). Feature vectors were constructed with indicators for individual strain modifications, with multi-valued modifications (like the core threonine operon specification) one-hot encoded for a total of 33 input dimensions and one output dimension (threonine yield). Hyperparameter searches were optimized for lowest mean absolute error. The resulting model used a batch normalization layer followed by seven feed-forward layers. The final layer configuration widths for the inner layers were 22, 19, 16, 13, 10, 7, 4. The initial activation function chosen was Soft max and the inner-layer activation function was Rectified Linear Unit. The mean error during cross-validation was 3.1% of the total output range. with an inter-quartile range equal to 1.0% of the total output range. The model output (predicted vs. actual) is displayed in FIG. 3. Because of the scarcity of training data with yields of 1.2 g/L or greater, mean absolute error increased at higher outputs—0.04457 for strains yielding less than 1.2 g/L, and 0.2106 for high-yielding strains.

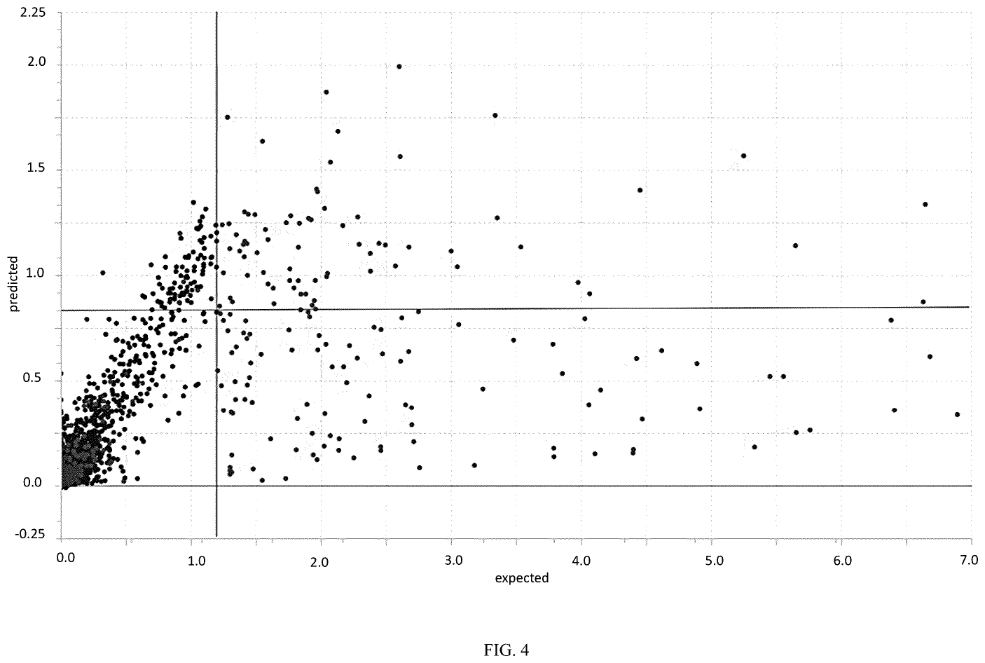

The next step was to expand model predictions to a much larger virtual space of modifications. There, some strains were expected to have threonine production outside of the range achieved in the training set. Since regressors typically do not extrapolate well, the model is expected not to produce accurate numeric predictions for strains yielding more than 2.6 g/L of threonine. To study this, a model was trained only with samples that produced less than 1.2 g/L, as shown in FIG. 4, and evaluated it with the holdout set of 430 samples, 173 of which had production levels greater than or equal to the cutoff of 1.2 g/L of the training set. Eighty of these high-producing samples were correctly picked by the model, a sensitivity of 44%. Only three low-producing samples in the holdout set were predicted to be high-producing, a false discovery rate of 3/(3+80) or 4%. High-producing samples in the holdout set showed very little correlation between the predicted and actual values (Pearson's coefficient of 0.36).

When the full model was run against all possible virtual samples, 1.3% of the samples are predicted to be in a high-producing category (above 1.2 g/L). Even if ⅔ of possible high producing variants were lost due to low sensitivity, the constructed deep learning model still gave 27,659 construct designs for the next round of strain engineering. Due to the low false discovery rate, one would expect over 24,000 of these designs to yield high-threonine producing strains.

### Example 8: AI-Driven Engineering: Construction and Testing of a Second Set of Threonine-Producing Bacterial Strains

In the next round of modeling, an exhaustive set of all possible combinations of features was generated and applied to predict threonine production. A filtering pre-processing was used to eliminate variants that were logically impossible, leaving 2,162,689 total variants. A second filter was used to remove strains that would be more complicated to engineer. In particular, the number of knockouts was limited to 3 or less, and variants with plasmids carrying the thr operon were excluded. A total of 178,561 of these constrained variants were submitted for prediction, 1,076 of which had predicted production higher than 1.2 g/L. 169 of these strains were engineered and tested. 37% of them demonstrated expected high yield (an enrichment of 30-fold over their overall 1.3% fraction in total virtual space). Of these high-producing variants, 17 produced more threonine compared with the industrial control strain NRRL B-21593, with the highest yield being 5.84 g/L (significantly higher than control strain), as shown in Table 4. 287 additional strains were constructed to provide data on the performance of specific combinations of modifications of gene expression “around” high producing variants suggested by Deep Learning.

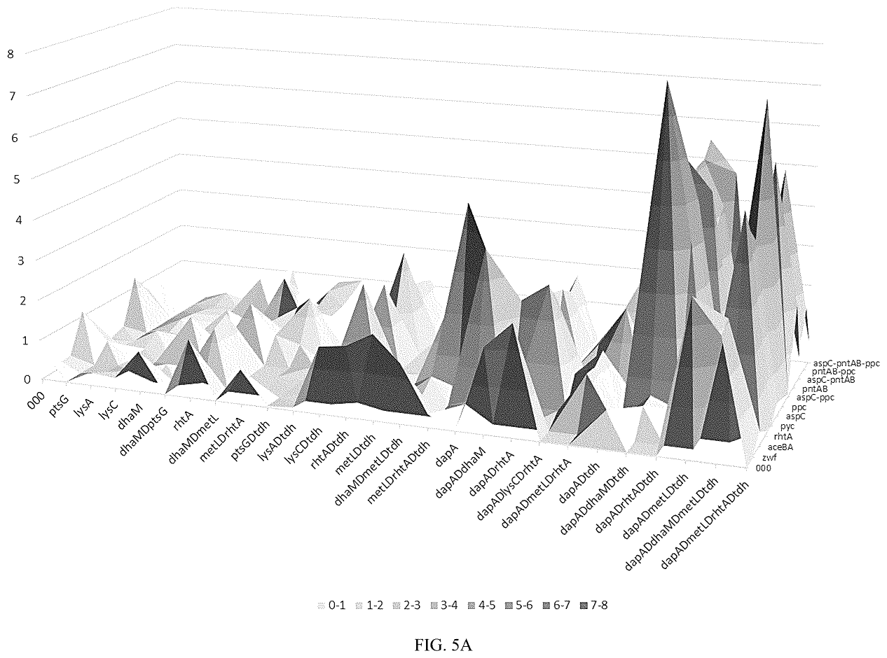

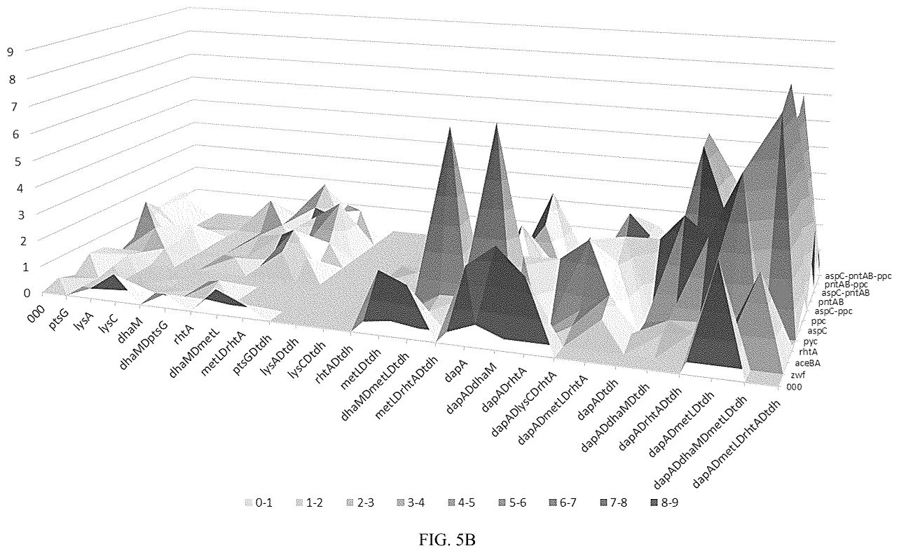

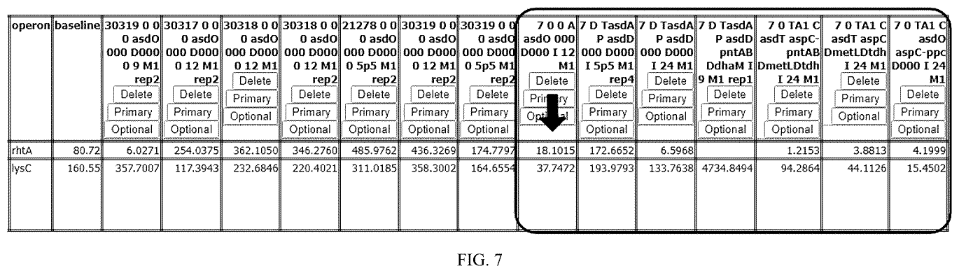

FIGS. 5A-5B show representations of threonine production values as a function of construction variables, (e.g., knockouts and overexpressed genes). All samples were shown as 2 groups: 7asd0 (FIG. 5A) and 7asdT (FIG. 5B) (see naming conventions outlined in Table 2). Displayed samples collected at 24 hours, threonine operon transcription induced by IPTG.

The following factors positively impacted threonine production:
1) upregulated asd gene;
2) various combinations of deletions of rhtA, dapA, dhaM, metL and tdh genes;
3) various combinations of overexpressed aspC, pntAB and ppc genes.

TABLE 4
Second set of engineered strains with highest threonine production.

| | avg g/L | max |
|---|---|---|
| Modification profile | (3 measurements) | g/L |
| 7_0_TA1_C_asdT_aspC-ppc_DdapADmetLDtdh_I_24_M1 | 5.8388 | 8.1743 |
| 7_0_TA1_C_asdT_aspC-pntAB-ppc_DdapADmetLDtdh_I_24_M1 | 5.8115 | 6.7463 |
| 7_0_TA1_C_asdT_aspC_DdapADmetLDtdh_I_24_M1 | 5.5267 | 7.6683 |
| 7_0_TA1_C_asdT_aspC-pntAB_DdapADmetLDtdh_I_24_M1 | 5.4387 | 9.8815 |
| 7_0_TA1_C_asdT_pntAB-ppc_DdapADmetLDtdh_I_24_M1 | 5.2725 | 9.8710 |
| 7_0_TA1_C_asdT_pntAB-ppc_DdhaMDmetLDtdh_I_24_M1 | 5.0008 | 6.8526 |
| 7_0_TA1_C_asdT_aspC-ppc_DdapADdhaM_I_24_M1 | 4.9839 | 6.1978 |
| 7_0_TA1_C_asdO_aspC_DdapADmetLDtdh_I_24_M1 | 4.7706 | 6.9526 |
| 7_0_TA1_C_asdT_aspC-ppc_DdhaMDmetLDtdh_I_24_M1 | 4.2474 | 8.7177 |
| 7_0_TA1_C_asdT_aspC-pntAB-ppc_DdapADdhaM_I_24_M1 | 4.2150 | 6,7913 |
| 7_0_TA1_C_asdO_pntAB_DdapADmetLDtdh_I_24_M1 | 4.1934 | 7.4352 |
| 7_0_TA1_C_asdO_pntAB-ppc_DdapADmetLDtdh_I_24_M1 | 4.0474 | 6.6999 |
| 7_0_TA1_C_asdT_aspC-pntAB_DdhaMDmetLDtdh_I_24_M1 | 4.0067 | 5,8086 |
| 7_0_TA1_C_asdTaspC-pntAB_DdapADdhaM_I_24_M1 | 3.9269 | 5.9511 |
| 7_0_TA1_C_asdT_000_DdapADmetLDtdh_I_24_M1 | 3.9127 | 3.9914 |
| 7_0_TA1_C_asdT_aspC-ppc_DdapADmetLDtdh_I_24_M1 | 5.8388 | 8.1743 |
| 7_0_TA1_C_asdT_aspC-pntAB-ppc_DdapADmetLDtdh_I_24_M1 | 5.8115 | 6.7463 |
| 7_0_TA1_C_asdT_aspC_DdapADmetLDtdh_I_24_M1 | 5.5267 | 7.6683 |
| 7_0_TA1_C_asdT_aspC-pntAB_DdapADmetLDtdh_I_24_M1 | 5.4387 | 9.8815 |
| 7_0_TA1_C_asdT_pntAB-ppc_DdapADmetLDtdh_I_24_M1 | 5.2725 | 9.8710 |
| 7_0_TA1_C_asdT_pntAB-ppc_DdhaMDmetLDtdh_I_24_M1 | 5.0008 | 6.8526 |
| 7_0_TA1_C_asdT_aspC-ppc_DdapADdhaM_I_24_M1 | 4.9839 | 6.1978 |
| 7_0_TA1_C_asdO_aspC_DdapADmetLDtdh_I_24_M1 | 4.7706 | 6.9526 |
| 7_0_TA1_C_asdT_aspC-ppc_DdhaMDmetLDtdh_I_24_M1 | 4.2474 | 8,7177 |
| 7_0_TA1_C_asdT_aspC-pntAB-ppc_DdapADdhaM_I_24_M1 | 4.2150 | 6.7913 |
| 7_0_TA1_C_asdO_pntAB_DdapADmetLDtdh_I_24_M1 | 4.1934 | 7.4352 |

Data shown in FIG. 5 highlight patterns in the effects of individual genes across many pairwise gene combinations. However, production peaks caused by specific combinations of engineered elements cannot be simply explained by mechanistic understanding or extrapolated from observed effect of individual genes or even tested combinations without AI assistance.

### Example 8: Third Round of Threonine-Producing Bacterial Strain Design

The theoretical yield of threonine conversion from glucose is 81% g/g (122% mole/mole (Lee, K. H., et al., Systems metabolic engineering of Escherichia coli for L-threonine production. Mol Syst Biol 2007. 3: p. 149.). Together with the production rate of a target molecule, yield is the most important property of an industrial strain. In the conditions tested, yield of the control industrial strains vary from 22 to 50%. Several of the most productive strains constructed in the study has glucose utilization yield of 24-28%. With the data on glucose utilization for strains engineered in the study (Table 4), Deep Learning optimization will be applied to improve this property.

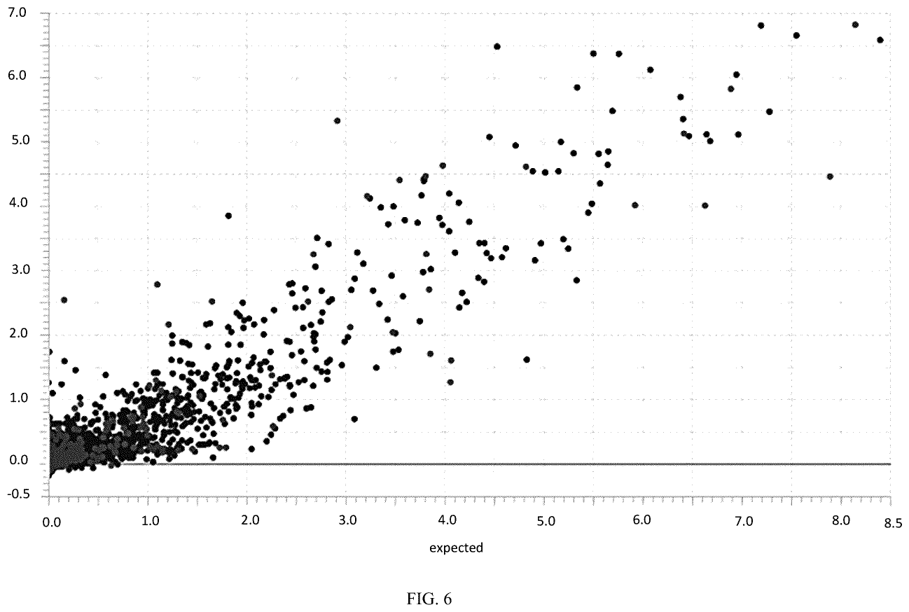

The deep learning regressor was retrained on the combined results of the first two rounds of strain engineering, raising average validation accuracy (classifying high- vs. low-producing variants) from 91% to 98%. This increase is likely due to the substantial enrichment of high-producing variants in the combined training set. The regressor was then run on a new set of 102841 candidate strains that differ from previously engineered strains by no more than one insert and/or knockout. 426 strains belonged to the high-producing category this time defined as 4 g/L (versus 1.2 g/L used in the first round). As expected, predicted yields plateaued around 9 g/L, which was a highest production value in the training set. These data are presented in Table 5 and FIG. 6.

TABLE 5
Prediction vs, actual threonine production levels for top predicted strains of third round of strain engineering.

| Modification profile | predicted | actual |
|---|---|---|
| 7_0_TA1_C_asdT_aspC-pntAB-ppc_DdapADmetLDrhtADtdh_I_24_M1 | 8.3058 | 2.3506 |
| 7_0_TA1_C_asdT_pntAB-ppc-pyc_DdapADdhaM_I_24_M1 | 8.1041 | 3.3506 |
| 7_0_TA1_C_asdT_aspC-ppc_DdapADmetLDrhtA_I_24_M1 | 7.6857 | 1.8031 |
| 7_0_TA1_C_asdT_aspC-ppc_DdapADdhaMDmetLDtdh_I_24_M1 | 7.6482 | 8.1468 |
| 7_0_TA1_C_asdT_aspC-ppc-pyc_DdapADdhaM_I_24_M1 | 7.3915 | 3.8510 |
| 7_0_TA1_C_asdT_aspC-pntAB-ppc_DdapADdhaMDtdh_I_24_M1 | 7.3396 | 4.7401 |
| 7_0_TA1_C_asdT_aspC-ppc_DdapADmetLDrhtADtdh_I_24_M1 | 7.1992 | 0.5489 |
| 7_0_TA1_C_asdT_ppc_DdapADlysCDrhtA_I_24_M1 | 7.1896 | 0.0000 |
| 7_0_TA1_C_asdT_aspC_ppc_DdapA_I_24_M1 | 7.1515 | 1.8157 |
| 7_0_TA1_C_asdT_aspC-pntAB-ppc_DdapADdhaMDmetLDtdh_I_24_M1 | 7.0501 | 6.9461 |

### Example 9: Expansion of Engineering Gene Repertoire by Metabolic Modeling and Machine Learning Analysis of Expression Data

To expand engineering repertoire beyond the initial set of genes selected by metabolic analysis, whole genome expression profiles of constructed strains were searched for additional genes which can increase threonine production. Using tools developed in the study and displayed at maseq.theseed.org, differential expression patterns were superimposed with various types of functional gene grouping, like PATRIC's subsystems (Davis, J. J., et al., The PATRIC Bioinformatics Resource Center: expanding data and analysis capabilities. Nucleic Acids Res. 2020. 48(D1); p. D606-D612.), Paulson's imodulons (Rychel, K., et al., iModulonDB; a knowledgebase of microbial transcriptional regulation derived from machine learning. Nucleic Acids Res, 2021. 49(D1); p. D112-D120.), and several others. Even though some interesting genes could be uncovered in such manual analysis, understanding multidimensional nature of Interactions effecting expression requires a systematic computational approach. Random Forest was capable of generating genes expression classifiers, which predicts high- or low-producing variant strains based on their expression changes. The challenge in using such classifiers to expand engineering repertoire (beyond classification purposes) is to separate genes which cause production changes from mere markers of production level. This requires additional “functional” filtering, for example, by metabolic analysis. Up- or down-regulated genes in strains producing threonine could be included in the engineering process if they: (1) belong to precursor pathways and transcriptional shift is caused by depletion of a final product; (2) bifurcate from threonine pathway or are a part of threonine degradation pathways; or (3) recruit the same cofactors. “Parasitic” expression changes, which complicate interpretation and should be ignored, are caused by growth stage effects, a general “overproduction shock” and other remote cascades of expression changes.

100 strains constructed in the project were subjected to RNA Seq analysis as described in Example 1. Six different random forest classifiers were trained with the RNA Seq data. The models used three different input sets: all genes, genes in subsystems, and genes in Paulsen's iModulons. For each input set, two different versions of the model were constructed and tested, one where the input values were the actual TPM numbers and one where the input values were (−1,0,1) depending on whether the TPM value was below the half mean, close to the mean, or twice the mean. The means were computed from 299 E. coli MG1655 samples from the SRA. The model cross-validation results are shown in Table 3. In each cell of the table, it shows the mean accuracy, the IQR (interquartile range), and the model's sensitivity to high-producing samples. See also FIG. 7.

TABLE 6
Cross-validation results for the six RNA seq expression data classifiers.

| | Trimean | Best | | Sensitivity | Sensitivity |
|---|---|---|---|---|---|
| | Accuracy | Accuracy | IQR | (best) | (combined) |
| All Genes, Raw pression | 0.70 | 0.75 | 0.13 | 0.00 | 0.00 |
| All Genes, Expression Categories | 0.70 | 0.75 | 0.10 | 0.33 | 0.07 |
| iModulon Genes, Raw Expression | 0.67 | 0.75 | 0.08 | 0.00 | 0.00 |
| iModulon Genes, Expression | 0.70 | 0.80 | 0.15 | 0.33 | 0.07 |
| Categories | | | | | |
| Subsystem Genes, Raw Expression | 0.69 | 0.75 | 0.20 | 0.33 | 0.36 |
| Subsystem Genes, Expression | 0.68 | 0.80 | 0.18 | 1.00 | 0.50 |
| Categories | | | | | |

When cross-validation was taken into account, the most accurate setup was the −/0/+ version for all genes, which had an IQR comparable to the most stable model (0.10 vs 0.08), but a higher mean accuracy (0.70 vs 0.67). The best model produced from this setup had an accuracy of 75% (about 5% higher than the mean of 70% shown in the table), with a sensitivity of 33% (it catches 33% of the high-producing samples) and a fallout of 0 (which means no false positives).

297 of the genes had an impact on the classifications. For the most impactful of these, the table below shows the gene name, the impact (as a reduction in entropy), the Pearson correlation between the expression and the threonine production, and the SEED functional assignment.

The impact is expressed as a reduction in entropy. The maximum entropy for this model is 1.585 (log₂ 3), so an impact of 0.0487 represents a 3% increase in knowledge about the threonine production categories of the inputs.

A Pearson correlation is computed on the raw expression and production values, not the categories. The important attribute of the Pearson correlation here is not the magnitude (which is an apples-to-oranges comparison), but the sign (which is shown in Table 7). The sign of the Pearson correlation indicates whether the gene promotes threonine (positive) or inhibits it (negative).

TABLE 7
Significant features, metal genes whose expression changes drive AI predictions.

| | % | | |
|---|---|---|---|
| gene | impact | dir | function |
| aceB | 2.0156 | − | Malate syntha.se (EC 2.3.3.9) |
| pabC | 1.3414 | + | Aminodeoxychorismate lyase (EC 4.1.3.38) |
| rib | 1.3375 | − | 3,4-dihydroxy-2-butanone 4-phosphate synthase (EC 4.1.99.12) |
| fadB | 1.2696 | + | Enoyl-CoA hydratase (EC 4.2.1.17)/Oelta(3kis.delta(2>,-trans-enoyl- |
| | | | CoA isomerase (EC 5.3.3.8)/3- |
| dapA | 1.2413 | − | 4-hydroxy-tetrahydrodipicolinate synthase (EC 4.3.3.7) |
| mntH | 1.2103 | + | Manganese transport protein MntH |
| IpxD | 1.1752 | − | UDP-3-0-(3-hydrosymyristoyl)glucosamine N-acyltransferase |
| | | | (EC 2.3.1.191) |
| fabZ | 1.0864 | − | 3-hydroxyacyl-[acyl<arrier-protein)dehydratase., FabZ form |
| | | | (EC 4.2.1.59) |
| fbaA | 1.0181 | − | Fructose♦biophosphate aldolaseclacss II (EC 4.1.2.13) |
| sdhB | 0.9882 | + | Succinate dehydrogenase iron-sulfur protein (EC 1.3.5.1) |
| sthA | 0.9822 | − | Soluble pyridine nucleotide transhydrogenase (EC 1.6.1.U |
| ftsW | 0.9569 | − | Peptidoglycan glycosyltransferase FtsW (EC 2.4.1.129) |
| Asd | 0.9453 | + | Asparate-semialdehyde dehydrogenase (EC 1.2.1.11) |
| atpG | 0.9295 | − | ATP synthasegammachain (EC 3.6.3.14) |
| gpmM | 0.9294 | + | 2,3-bisphosphoglycerate-independent phosphoglycerate mutase |
| | | | (EC 5.4.2.12) |
| kdsA | 0.922 | − | 2-Keto-3-deoxy-D-manno□ctulosonate-8-phosphate synthase |
| | | | (EC 2.5.1.55) |
| ItaE | 0.9162 | + | Low-specificity L-threonine aldolase (EC 4.12.48) |
| purH | 0.8944 | − | IMP cyclohydrolase (EC 3.5.4.10)/ |
| | | | Phosphoribosylaminoimidazolecarboxamide formyltransferase |
| tpiA | 0.8663 | − | Triosephosphate isomerase (EC 5.3.1.1) |
| murI | 0.7991 | − | Glutamate racemase (EC 5.1.1.3) |
| pvkF | 0.7922 | − | Pyruvate kinase (EC 2.7.140) |
| fabD | 0.7811 | − | Malonyl CoA-acyl carrier protein transacylase (EC 2.3.1.39) |
| tdh | 0.7741 | + | L-threonine 3-dehydrogenase (EC 1.1.1.103) |
| fbp | 0.7521 | + | Fructose-1.6-bisphosphatease, type I (EC 3.1.3.11) |
| glpF | 0.7486 | − | Glycerol update facilitator protein |
| atpF | 0.7288 | − | ATP synthase F0 sector subunit b (EC 3.6.3.14) |
| gpmA | 0.7145 | − | Phosphoglycerate mutase (EC 5.4.2.11) |
| atpC | 0.7115 | − | ATP synthase epsilon chan (EC 3.6.3.14) |
| eno | 0.6962 | − | Enolase (EC 4.2.1.11) |
| gpsA | 0.6908 | − | Glycerol-3-phosphate dehydrogenase [NAD(P)+] (EC 1.1.1.94) |
| crr | 0.6907 | − | PTS system, glucose-specific IIA component (EC 2.7.1.199) |
| gltB | 0.6798 | − | Glutamate synthase [NADPH]large chain (EC 1.4.1.13) |
| hemB | 0.6776 | − | Uroporphyrinogen III decarboxylase (EC 4.1.1.37) |
| cysE | 0.6716 | − | Serine acetyltransferase (EC 2.3.1.30) |
| nrdD | 0.6662 | + | Ribonucleotide reductase of class III (anaerobic), large subunit |
| | | | (EC 1.17.4.2) |
| ubiG | 0.6572 | + | 3-demethylubiquinol 3-O-methyltransferase (EC 2.1.1.64) @ |
| | | | 2-polyprenyl-6-hydroxyphenyl meth |
| bioB | 0.6465 | + | Biotin synthase (EC 2.8.1.6) |
| ilvC | 0.6364 | − | Ketol-acid reductoisomerase (NADP(+)) (EC 1.1.1.86) |

Metabolic genes with clearly defined functions, as well as more obscure genes including hypotheticals were among strong predictors of threonine production. Because of the redundant nature of the expression signal, the analysis was limited to metabolic genes only. Metabolic modeling of the impact of the changes in expression of these genes was applied to select additional gene candidates to include in subsequent strain engineering.

### Example 10. Additional AI-Driven Ranking of genes from Expression Signatures of High Threonine Production Strains

The goal of the following analysis was to find individual genes or small collections of genes that are most able to predict high-threonine production using a different computational strategy. Using the same 100 RNA Seq samples, all genes with RNA-seq measurements (regardless of groupings like subsystems) were analyzed individually for prediction ability. In this analysis, rather than using large collections of genes derived from sets like subsystems, the genes were taken one at a time. For each gene, the expression level for each sample was used as input, and the production level (None, Low, High as before) was used as output. The 100 samples were randomly split into sets of 70% training and 30% testing, with equal proportions of each production class, and used to train a Random Forest model. For each gene, 10 random splits were chosen to train 10 individual Random Forest models, and the resulting accuracies were averaged together to help account for the small number of samples.

This process was repeated in three other ways. First, SMOTE (Synthetic Minority Oversampling Technique) was as an additional method for combating the low number of samples. This method brings the underrepresented classes to the same number of samples as the most represented class by generating synthetic data. Synthetic data points were only generated for the training set, such that the testing set remained composed of entirely real data. The two remaining runs of this data analysis were a repeat of the previous two (with and without SMOTE), but combined the None and Low classes of production into a single class representing all Low production samples.

From these four methods, a ranking of each gene was created based on the accuracy of the model at predicting the High class of production (in the binary case, this would be the recall). The four rankings from the models were used to produce a final ranking of each gene's expression data at predicting high producing strains. This was done with a weighted combination of ranks, simply by summing the index that each gene appeared in the four original rankings. Since the goal of this analysis was to investigate the highest-ranking genes, this weighted combination was chosen as it places importance on consistent high-ranking position across all four original lists. The final ranking of the top SO genes is shown in Table 8.

TABLE 8
Top 50 (high-impact) genes
predicted to produce high-threonine-
producing bacterial strains.

| Gene | Score |
|---|---|
| gpmM.3712 | 0 |
| bioC.786 | 15 |
| aceA.4101 | 18 |
| frdA.4255 | 24 |
| fecA.4392 | 41 |
| IpxL.1070 | 44 |
| gltX.2475 | 44 |
| bioA.783 | 48 |
| tonB.1277 | 52 |
| cysG.3446 | 58 |
| adhE.1265 | 76 |
| iscA.2608 | 83 |
| gshA.2763 | 83 |
| ribB.3122 | 84 |
| glnA.3959 | 84 |
| gltD.3293 | 91 |
| dadA.1209 | 111 |
| yeeO.2041 | 111 |
| mhpF.355 | 122 |
| yrfG.3476 | 138 |
| fabG.1110 | 141 |
| hemE.4085 | 146 |
| atpA.3836 | 149 |
| purE.530 | 152 |
| aceE.114 | 152 |
| menB.2334 | 161 |
| yggF.3011 | 168 |
| glyA.2631 | 169 |
| pfkA.4010 | 171 |
| gutQ.2780 | 171 |
| speB.3017 | 179 |
| ppc.4050 | 185 |
| glpK.4020 | 193 |
| thrB.3 | 199 |
| yfbQ.2363 | 202 |
| fabI.1317 | 208 |
| tpiA.4013 | 210 |
| ribE.422 | 210 |
| acpP.1111 | 211 |
| fbaB.2155 | 211 |
| aldB.3685 | 224 |
| aroG.762 | 231 |
| maeB.2538 | 234 |
| ubiE.3925 | 240 |
| thrA.2 | 243 |
| hyfD.2560 | 257 |
| ackA.2370 | 259 |
| atpC.3833 | 260 |
| tdcD.3196 | 260 |

When metabolic analysis was applied to these genes, substantial agreement between the high-impact genes predicted from this RNA-seq analysis and the potentially beneficial knockouts and over-expressions predicted by the earlier metabolic modeling analyses was observed. Specifically, sixteen of the deletions and 43 of the overexpressions proposed by previous models were associated with one or more of the 317 genes that were identified as high impact from this RNA-seq analysis. Therefore, 46% of reactions predicted by metabolic modeling for the M_0_TA1_C_asdT base strain were also supported by this RNAseq data analysis. Of these, 12 reactions (3 knockdown/out, 9 overexpression) had high RNAseq impact scores of >0.5. These include deletion of: glcA, dapA, and hemE and overexpressions in: fadB, dapA, asd, purH, purK which may have positive effects on threonine production. The 7_0_TA1_C_asdT base strain had 10 of the 16 suggested reactions overlapping with impactful RNAseq reactions, of those 4 also overlapped with the M_0_TA1_C_asdT base strain results. These include reactions with high RNAseq impact scores in threonine biosynthesis as well asd, aceB and glcB. Of all these predictions, dapA, asd, and aceB have already been tested experimentally.

Impactful features represented in Table 7 and Table 8 are not identical but have a substantial overlap. Table 9 shows some of the top candidate genes for engineering supported by both metabolic modeling and RNAseq data. To filter gene candidates for further strain improvement, metabolic context analysis was applied, as described below.

TABLE 9
Top genes predicted by metabolic modeling to increase threonine production that arealso supported by RNAseq impact analysis.

| | | | | | | | RNAseq | |
|---|---|---|---|---|---|---|---|---|
| | BIGG | Mutation | | Predicted | Predicted | Predicted | impact | |
| Gene ID | reaction ID | Type | Base Flux | Flux | Threonine | Biomass | score | Base Strain |
| aceB/glcA | MALS | over- | 0.93259275 | 1.865185491 | 0.518160803 | 0.04944358 | 2.0156 | 7_0_TAI_C_asdT |
| | | expression | | | | | | |
| asd | ASAD | over- | −0.278047 | −0.556093986 | 0.489435657 | 0.059648599 | 0.9433 | 7_0_TAI_C_asdT |
| | | expression | | | | | | |
| thrA | ASPK | over- | 0.27804699 | 0.556093986 | 0.489435671 | 0.059648602 | 0.4436 | 7_0_TAI_C_asdT |
| | | expression | | | | | | |
| thrA | HSDy | over- | −0.2535737 | −0.50714741 | 0.426787393 | 0.061898899 | 0.3305 | 7_0_TAI_C_asdT |
| | | expression | | | | | | |
| serC | PSERT | knockdown | 0.12087669 | 0.060438346 | 0.262662948 | 0.066892052 | 0.3305 | 7_0_TAI_C_asdT |
| thrB | HSK | over- | 0.24 | 0.48 | 0.48 | 0.061482777 | 0.29 | 7_0_TAI_C_asdT |
| | | expression | | | | | | |
| thrC | THRS | over- | 0.24 | 0.48 | 0.48 | 0.061482777 | 0 | 7_0_TAI_C_asdT |
| | | expression | | | | | | |
| ompC/ompF | THRtex | over- | −0.2155305 | −0.431061029 | 0.446485023 | 0.062570464 | 0 | 7_0_TAI_C_asdT |
| | | expression | | | | | | |
| ompC/ompF | HOMtex | knockout | −0.0135737 | 0 | 0.253573705 | 0.067 | 0 | 7_0_TAI_C_asdT |
| ompC/ompF | NH4tex | knockout | 1.55288157 | 0 | 0.253573705 | 0.040649171 | 0 | 7_0_TAI_C_asdT |
| fadB | CTECOA18 | over- | −0.0058647 | −0.011729369 | 0.062485291 | 0.241216 | 1.2696 | M_0_TAI_C_asdT |
| | | expression | | | | | | |
| fadB | ECOAH8 | over- | 0.00586468 | 0.011729369 | 0.062485291 | 0.241216 | 1.2696 | M_0_TAI_C_asdT |
| | | expression | | | | | | |
| fadB | HACD8 | over- | 0.00586468 | 0.011729369 | 0.062485291 | 0.241216 | 1.2696 | M_0_TAI_C_asdT |
| | | expression | | | | | | |
| fadB | HACD6 | over- | 0.03663031 | 0.073260618 | 0.049884153 | 0.137528434 | 1.2696 | M_0_TAI_C_asdT |
| | | expression | | | | | | |
| fadB | ECOAH6 | over- | 0.03663031 | 0.073260618 | 0.049884153 | 0.137528434 | 1.2696 | M_0_TAI_C_asdT |
| | | expression | | | | | | |
| dapA | DHDPS | knockdown | 0.05415375 | 0.027076875 | 0.043520037 | 0.15 | 1.2413 | M_0_TAI_C_asdT |
| asd | ASAD | over- | −0.0941538 | −0.1883075 | 0.1207904 | 0.147233941 | 0.9453 | M_0_TAI_C_asdT |
| | | expression | | | | | | |
| purH | AICART | over- | 0.07972185 | 0.1594437 | 0.060778259 | 0.13929864 | 0.8944 | M_0_TAI_C_asdT |
| | | expression | | | | | | |
| purH | IMPC | over- | −0.0797218 | −0.1594437 | 0.060778242 | 0.139298645 | 0.8944 | M_0_TAI_C_asdT |
| | | expression | | | | | | |
| hemE | UPPDCI | knockdown | 6.69E−05 | 3.35E−05 | 0.043521502 | 0.002680275 | 0.6776 | M_0_TAI_C_asdT |
| glyA | GHMT2r | knockout | 0.15221735 | 0 | 0.187185779 | 0.144024447 | 0.5902 | M_0_TAI_C_asdT |
| purK | AIRC2 | over- | 0.06591345 | 0.1318269 | 0.057100477 | 0.140702372 | 0.5794 | M_0_TAI_C_asdT |
| | | expression | | | | | | |
| purB | ASDL2r | over- | 0.06591345 | 0.1318269 | 0.057100463 | 0.140702371 | 0.4835 | M_0_TAI_C_asdT |
| | | expression | | | | | | |
| thrA | ASPK | over- | 0.09415375 | 0.1883075 | 0.120790407 | 0.147233941 | 0.4436 | M_0_TAI_C_asdT |
| | | expression | | | | | | |
| menB | DHNCOAS | over- | 6.69E−05 | 0.0001338 | 0.0769759 | 0.3 | 0.4101 | M_0_TAI_C_asdT |
| | | expression | | | | | | |
| purL | PRFGS | over- | 0.06591345 | 0.1318269 | 0.057127894 | 0.140701312 | 0.4101 | M_0_TAI_C_asdT |
| | | expression | | | | | | |
| menA | DHNAOT4 | over- | 6.69E−05 | 0.0001338 | 0.0769759 | 0.3 | 0.3919 | M_0_TAI_C_asdT |
| | | expression | | | | | | |
| metK | METAT | knockdown | 0.00124755 | 0.000623775 | 0.042381722 | 0.068485421 | 0.343 | M_0_TAI_C_asdT |
| serC | OHPBAT | over- | 3.35E−05 | 6.69E−05 | 0.0769759 | 0.3 | 0.3305 | M_0_TAI_C_asdT |
| | | expression | | | | | | |

Overexpression = base flux *2, knockdown = base flux/2, knockout = 0 flux.

Previous modeling analyses were designed to predict the impact of individual modifications and combinations of modifications. Here, the model was applied to attempt to mechanistically understand the three most successful threonine production strains; M_0_TA1_C_asdT_pntAB-aspC_DtdhDmetLDdapA, M_0_TA1_C_asdT_pntAB-ppc_DtdhDmetLDdapA, and M_0_TA1_C_asdT_pntAB-ppc_DthtADdapA (See Table 5). Unlike in previous studies, models were fitted to the observed growth rate and threonine production rate for these strains, while also simulating as many of the modifications for these strains as possible. If the simulation of a modification prevents the model from attaining the observed growth and production rates, the modification was relaxed.

The over-expression of pntAB utilized in the three most productive strains was essential for the increased threonine production, in that the flux through the reaction associated with this gene had to increase dramatically to achieve the observed threonine production rate by increasing production of the NADPH cofactor that is consumed significantly in the threonine pathway. Half of the reactions associated with the aspC overexpression increased in flux, and this directly increased threonine by targeting genes in the threonine biosynthesis pathway. The ppc gene, involved in carbon rearrangement, was difficult for the model to simulate.

The models predicted that the dapA knockouts included in the most productive strains had little impact on threonine production. The models reported that it was detrimental to threonine production to knockout this gene entirely, however knockdown or overexpression of this gene had little effect on threonine otherwise. The pathway associated with this gene could still carry significant flux without reducing the observed threonine production or growth (even with glucose uptake restricted to just the value needed to support the observed growth and threonine production). Models were unable to predict the effect of the rhtA mutant because it had no flux in the base strain. Models also predicted flux through tdh, a gene involved in threonine catabolism would increase flux through threonine production, however it is knocked out for practical reasons in most mutants to reduce threonine degradation and constrained in the models for predictions.

Only one of the modifications entirely defied prediction by the models—deletion of metL. In the model, metL is one of three genes associated with the first step of the threonine biosynthesis pathway. Thus, a knockout of this gene is expected to reduce flux through this pathway. Yet, this knockout was essential in experimental studies for achieving significant threonine production in two of the most productive strains.

Overall, the modeling studies reported here demonstrate both the strengths and limitations associated with the application of metabolic models to aid in guiding metabolic engineering efforts. With individual knockouts and over-expressions, the models showed excellent qualitative agreement (77%) with experimental results, but this declined dramatically to 46% when attempting to predict the combined impact of multiple modifications. Further, basic FBA modeling with standard constraints performed poorly at qualitatively predicting the impact of modifications on growth and threonine production rates (more sophisticated methods available likely would have performed better). This demonstrates that models perform well at qualitatively predicting impacts of small perturbations, but struggle with larger perturbations. Interestingly, extensive synergy was observed between modeling and machine learning approaches. Machine learning was able to predict the impact of combinations of knockouts and over-expressions with great accuracy, and thus machine learning was able to propose particularly effective combinations to attempt, leading ultimately to substantially improved threonine-producing bacterial strains. Also, when applying machine learning to identify particularly impactful new knockouts and over-expressions based on RNA-seq data, significant agreement was found between these predictions and predictions by the models. The most effective knockouts and over-expressions will be those that were identified by both the machine learning and the modeling approaches. Finally, the value of models in testing and validating a mechanistic understanding of the most productive strains. Models can explain many of the modifications integrated into these strains, but in some cases, as in metL, modifications defied mechanistic understanding. This is valuable however as it points to gaps in the mechanistic understanding of these systems and metabolism in general, and if these gaps can be filled, it will improve modeling predictions in the future.

### Example 11. MetL Deletion and Threonine Production

The present Example illustrates the surprising and unexpected finding that the deletion of metL results in increased threonine production.

The ATCC21277B E. coli strain was used as the background strain in the experiments described in the present Example. The ATCC21277B E. coli strain comprises a threonine operon under the control of a tac promoter. The threonine operon was present in the chromosome at its native location, with a feedback-resistant thrA gene. The asd gene was also present on the chromosome, and under the control of the tac promoter. The genes listed on the plasmid were controlled by a constitutive promoter (J23108 promoter). The cells were grown in 96 deep well plates in minimal media, before being assayed for threonine production as described above.

As shown in Table 10, deletion of tdh and metL resulted in the generation of a bacterial strain capable of producing threonine at a level greater than that when tdh was deleted alone. This was surprising given that metL is homologous to thrA, and codes for the same enzymatic activities (aspartokinase and homoserine dehydrogenase).

TABLE 10
Threonine production by strain with genetic modifications of interest.

| | | | Threonine |
|---|---|---|---|
| | | Threonine production | production |
| Strain | Genes on plasmid | (induced) | (un-induced) |
| ATCC21277B Δtdh | | 1.7 g/L threonine | 0.7 g/L threonine |
| ATCC21277B Δtdh ΔmetL | | 2.3 g/L threonine | 1.0 g/L threonine |
| ATCC21277B Δtdh ΔmetL | ppc | 2.1 g/L threonine | 0.5 g/L threonine |
| ATCC21277B Δtdh ΔmetL | aspC | 2.6 g/L threonine | 0.9 g/L threonine |
| ATCC21277B Δtdh ΔmetL | pntAB | 2.8 g/L threonine | 1.1 g/L threonine |
| ATCC21277B Δtdh ΔmetL | pntABppc | 2.8 g/L threonine | 1.3 g/L threonine |
| ATCC21277B Δtdh ΔmetL | pntABaspC | 3.1 g/L threonine | 0.9 g/L threonine |
| ATCC21277B Δtdh ΔmetL | ppcaspC | 2.9 g/L threonine | 0.8 g/L threonine |
| ATCC21277B Δtdh ΔmetL | pntABaspCppc | 3.3 g/L threonine | 1.0 g/L threonine |
| ATCC21277B Δtdh ΔmetL ΔdapA | | 3.9 g/L threonine | 1.3 g/L threonine |
| ATCC21277B Δtdh ΔmetL ΔdapA | aspC | 5.5 g/L threonine | 1.6 g/L threonine |
| ATCC21277B Δtdh ΔmetL ΔdapA | pntAB | 3.2 g/L threonine | 1.4 g/L threonine |
| ATCC21277B Δtdh ΔmetL ΔdapA | pntABppc | 5.3 g/L threonine | 2.1 g/L threonine |
| ATCC21277B Δtdh ΔmetL ΔdapA | pntABaspC | 5.4 g/L threonine | 1.9 g/L threonine |
| ATCC21277B Δtdh ΔmetL ΔdapA | ppcaspC | 5.8 g/L threonine | 1.9 g/L thirconine |
| ATCC21277B Δrdh ΔmetL ΔdapA | pntABaspCppc | 5.8 g/L threonine | 1.4 g/L threonine |
| ATCC21277B Δtdh ΔmetL ΔdhaM | | 1.9 g/L threonine | |
| ATCC21277B Δtdh ΔmetL ΔdhaM | ppc | 3.1 g/L threonine | 0.6 g/L threonine |
| ATCC21277B Δtdh ΔmetL ΔdhaM | aspC | 2.7 g/L threonine | 0.6 g/L threonine |
| ATCC21277B Δtdh ΔmetL ΔdhaM | pntABppc | 5.0 g/L threonine | 0.9 g/L threonine |
| ATCC21277B Δtdh ΔmetL ΔdhaM | pntABaspC | 4.0 g/L threonine | 1.0 g/L threonine |
| ATCC21277B Δtdh ΔmetL ΔdhaM | ppcaspC | 4.2 g/L threonine | 1.0 g/L threonine |
| ATCC21277B Δtdh ΔmetL ΔdhaM | pntABaspCppc | 3.2 g/L threonine | 0.9 g/L threonine |

The mechanism for metL-deletion-dependent enhancement of threonine production remains unexplained. It is also unclear how and why some combinations of deletions and supplementations (e.g., gene overexpressed on a plasmid) produce threonine at higher levels than do others.

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be. incorporated by reference in its entirety. Definitions that are contained in text incorporated by reference are excluded to the extent that they contradict definitions in this disclosure.

Other embodiments are set forth in the following claims.

What is claimed is:

1. A method of engineering a target-biomolecule-producing bacterial cell, the method comprising:
(i) identifying a set of optimized parameters predicted to result in increased production of the target biomolecule by the bacterial cell;
(ii) constructing a plurality of bacterial strains, each bacterial strain comprising one or more of the optimized parameters of the set of optimized parameters identified in (i);
(iii) collecting target biomolecule production data from the strains constructed in (ii);
(iv) performing a computational analysis of the data collected in (iii) in order to obtain a further optimized set of parameters that predict increased production of the target biomolecule.
(v) repeating steps (ii), (iii), and (iv); and
(vi) constructing one or more final bacterial strains, each bacterial strain comprising one or more of the optimized parameters of the set of optimized parameters identified in (i) or (iv).

2. The method of claim 1, wherein the bacterial cell comprises a modified operon comprising a gene sequence encoding the target biomolecule.

3. The method of claim 2, wherein the modified operon is operably linked to a non-native promoter.

4. The method of claim 1, wherein the parameters are selected from the group consisting of host strain, inactivated genes, and overexpressed genes.

5. The method of claim 1, wherein the step of repeating is performed at least twice.

6. The method of claim 1, wherein the computational analysis comprises machine learning (ML).

7. The method of claim 6, wherein the computational analysis further comprises metabolic modeling (MM).

8. The method of claim 1, wherein the parameters further comprise:
(i) presence of endogenous operon comprising the gene sequence encoding the target biomolecule;
(ii) chromosomal or plasmid localization of the modified operon;
(iii) induction of the modified operon by Isopropyl β-D-1-thiogalactopyranoside (IPTG);
(iv) growth time post-induction; and
(v) culture medium type.

9. The method of claim 1, wherein the step of collecting further comprises collecting one or more of bacterial cell growth rate data, sugar conversion data, and RNA-Seq data.

10. The method of claim 1, wherein the bacterial cell is a bacterial cell of the strain ATCC 21277.

11. An engineered bacterial cell capable of producing threonine, wherein the engineered bacterial cell comprises a chromosome comprising a metL deletion.

12. The engineered bacterial cell of claim 11, wherein the engineered bacterial cell is an E. coli cell.

13. The engineered bacterial cell of claim 12, wherein the E. coli cell is an E. coli cell of the strain ATCC 21277.

14. The engineered bacterial cell of claim 11, wherein the engineered bacterial cell comprises an attenuated metL gene.

15. The engineered bacterial cell of claim 11, wherein the engineered bacterial cell comprises a chromosome comprising a deletion of one or more of tdh, dapA, and dhaM.

16. The engineered bacterial cell of claim 15, wherein the engineered bacterial cell comprises a plasmid comprising a nucleotide sequence encoding a ppc gene.

17. The engineered bacterial cell of claim 15, wherein the engineered bacterial cell comprises a plasmid comprising a nucleotide sequence encoding an aspC gene.

18. The engineered bacterial cell of claim 15, wherein the engineered bacterial cell comprises a plasmid comprising a nucleotide sequence encoding a pntAB gene.

19. The engineered bacterial cell of claim 15, wherein the engineered bacterial cell comprises one or more plasmids, each plasmid comprising one or more nucleotide sequences encoding one or more of a ppc gene, an aspC gene, and a pntAB gene.

20. The engineered bacterial cell of claim 15, wherein the engineered bacterial cell comprises a chromosome comprising at least two copies of one or more genes selected from ppc, aspC, and pntAB, thereby promoting overexpression of the one or more genes.

21. The engineered bacterial cell of claim 15, wherein the engineered bacterial cell comprises a chromosome comprising one or more of: (i) a ppc gene operably linked to a non-native promoter; (ii) an aspC gene operably linked to a non-native promoter; and (iii) a pntAB gene operably linked to a non-native promoter.

22. The engineered bacterial cell of claim 21, wherein the non-native promoter is a tac promoter.
